SEQUENCE LISTING (34 sequences)

SEQ ID NO: 1 - DNA, 2979 bp, Homo sapiens
atgcggagcaggagcaattccggggtccgcttggacgggtacgcgcggctggtgcagcaaaccatcctgtgttaccagaatcccgtcacggggctgctgtcagccagccatgagcagaaggatgcctgggtgcgggataacatctacagtatcctggccgtgtggggcctgggcatggcctaccgtaagaatgcagaccgcgatgaggacaaggccaaggcctacgagctggagcagaacgtggtgaagctgatgcgaggtcttctccagtgcatgatgagacaggtggccaaagtggagaagttcaaacacactcagagcaccaaggacagcctgcacgccaagtacaacaccgccacctgtggcacggtggtgggcgacgaccagtggggccacctccaggtggatgccacctctctcttcctcctgttcctggcccagatgaccgcctcaggcttacgtatcattttcactctcgatgaggtggccttcatacagaatcttgtcttttacatagaagctgcatataaagtcgctgattatggaatgtgggagcgtggagataagactaatcagggcatcccggaattgaatgcaagctccgtaggaatggccaaggcagctcttgaggcaattgatgaactggacctttttggagcccatggaggacgcaagtcagtgattcatgttctgccagatgaggtcgagcactgccagtctattctgttctccatgctgccaagagcgtcgacatctaaagaaattgatgctggacttctttccattatttccttcccggcctttgcagtggaagatgtaaaccttgtaaatgtgaccaaaaatgaaattatttctaagctccaggggcgttatggatgctgtcgcttccttcgagatggttataaaactccaagagaggaccctaatcgactgcattatgaccctgctgaactcaagctcttcgaaaacattgaatgtgagtggcctgtgttttggacatattttataatagatggagtcttcagtggtgatgctgttcaggtccaagaataccgagaggccctggagggaatactcatcagaggcaagaatgggatccgcctggtgcctgaactctacgctgtcccgcctaacaaggtagatgaagagtacaagaatcctcacacagtagaccgagttcctatggggaaggtgcctcatctgtggggccaatccttgtacatcctcagctcgctgttggcagagggattccttgccgctggtgaaatcgatcccttaaatagaagattttccacttcagtcaaacctgatgttgtagtacaagttactgttttggcagaaaacaatcacattaaggacttattgaggaaacacggggtgaacgtccagagtatcgcggacattcatccaattcaagtccagccgggccggattcttagtcacatatatgccaagcttggacggaataagaatatgaatttgagtgggcgaccgtatcgacatattggtgtccttggaacctctaaactatatgtgattaggaaccaaatctttacttttacaccccagttcaccgaccagcatcacttctacctggccctcgacaatgagatgatcgtggagatgctaaggatcgagctggcctacctgtgcacctgctggaggatgacgggcagacccacactcaccttccccatcagtcgcaccatgctcacaaatgatggctcagacattcattctgctgtgctctccacaattagaaaactagaggatggatattttggaggagccagagtaaaattagggaacctttcggaatttctcaccacatcgttctacacatatctgacttttctggatccagactgtgatgagaagttgtttgacaatgccagcgaagggactttcagtcctgatagtgattcagatttggtaggatatctggaagacacctgtaatcaagaaagccaagacgaacttgaccattatatcaaccaccttctgcaaagcacatcgttgaggtcctatctgcctcctctttgtaagaacacagaagaccgccatgtcttcagtgctatccactccacgcgggacatactttctgtgatggcaaaagcaaagggtttggaagttccatttgttcccatgactttgccgactaaagttctaagtgcccaccgtaaatcactgaatcttgttgattctcctcagccactcctagaaaaggttcctgaaagtgactttcagtggcccagagatgaccatggtgacgtggactgtgagaagctggttgagcagctaaaagattgttcgaacctacaggaccaagcagacattctgtacattctttatgtcataaagggtcccagctgggacacaaatctctctggacagcacggggtcaccgttcaaaaccttcttggtgagctctatgggaaagccggcttgaaccaggagtggggtctgattcgctacatctcaggccttctcaggaagaaagtggaggtcctggctgaggcctgcacagacctgctttcgcaccagaagcagctcaccgtgggcctgccgcccgagccccgggagaagatcatctctgcgccccttcccccagaggagctcacaaaactcatctacgaggccagtgggcaggacatcagcattgccgtcctcacgcaggagattgtggtttacctggccatgtatgtcagggcgcagcccagcctctttgtggagatgctgagactccggattggactgatcattcaggtgatggccacggagctggcacggagcctgaactgctcaggagaagaggcttctgaaagtttgatgaacctcagccctttcgatatgaaaaatctcctgcaccatattctaagtgggaaagagtttggcgttgaaagaagtgaggtccagcaccccatcctcgcccactggcacgtcatcctcagactcgggaggacatcacatcggctgggg

SEQ ID NO: 2 - protein, 993 aa, Homo sapiens
MRSRSNSGVRLDGYARLVQQTILCYQNPVTGLLSASHEQKDAWVRDNIYSILAVWGLGMAYRKNADRDEDKAKAYELEQNVVKLMRGLLQCMMRQVAKVEKFKHTQSTKDSLHAKYNTATCGTVVGDDQWGHLQVDATSLFLLFLAQMTASGLRIIFTLDEVAFIQNLVFYIEAAYKVADYGMWERGDKTNQGIPELNASSVGMAKAALEAIDELDLFGAHGGRKSVIHVLPDEVEHCQSILFSMLPRASTSKEIDAGLLSIISFPAFAVEDVNLVNVTKNEIISKLQGRYGCCRFLRDGYKTPREDPNRLHYDPAELKLFENIECEWPVFWTYFIIDGVFSGDAVQVQEYREALEGILIRGKNGIRLVPELYAVPPNKVDEEYKNPHTVDRVPMGKVPHLWGQSLYILSSLLAEGFLAAGEIDPLNRRFSTSVKPDVVVQVTVLAENNHIKDLLRKHGVNVQSIADIHPIQVQPGRILSHIYAKLGRNKNMNLSGRPYRHIGVLGTSKLYVIRNQIFTFTPQFTDQHHFYLALDNEMIVEMLRIELAYLCTCWRMTGRPTLTFPISRTMLTNDGSDIHSAVLSTIRKLEDGYFGGARVKLGNLSEFLTTSFYTYLTFLDPDCDEKLFDNASEGTFSPDSDSDLVGYLEDTCNQESQDELDHYINHLLQSTSLRSYLPPLCKNTEDRHVFSAIHSTRDILSVMAKAKGLEVPFVPMTLPTKVLSAHRKSLNLVDSPQPLLEKVPESDFQWPRDDHGDVDCEKLVEQLKDCSNLQDQADILYILYVIKGPSWDTNLSGQHGVTVQNLLGELYGKAGLNQEWGLIRYISGLLRKKVEVLAEACTDLLSHQKQLTVGLPPEPREKIISAPLPPEELTKLIYEASGQDISIAVLTQEIVVYLAMYVRAQPSLFVEMLRLRIGLIIQVMATELARSLNCSGEEASESLMNLSPFDMKNLLHHILSGKEFGVERSEVQHPILAHWHVILRLGRTSHRLG

SEQ ID NO: 3 - DNA, 1620 bp, Homo sapiens
atgcggagcaggagcaattccggggtccgcttggacgggtacgcgcggctggtgcagcaaaccatcctgtgttaccagaatcccgtcacggggctgctgtcagccagccatgagcagaaggatgcctgggtgcgggataacatctacagtatcctggccgtgtggggcctgggcatggcctaccgtaagaatgcagaccgcgatgaggacaaggccaaggcctacgagctggagcagaacgtggtgaagctgatgcgaggtcttctccagtgcatgatgagacaggtggccaaagtggagaagttcaaacacactcagagcaccaaggacagcctgcacgccaagtacaacaccgccacctgtggcacggtggtgggcgacgaccagtggggccacctccaggtggatgccacctctctcttcctcctgttcctggcccagatgaccgcctcaggcttacgtatcattttcactctcgatgaggtggccttcatacagaatcttgtcttttacatagaagctgcatataaagtcgctgattatggaatgtgggagcgtggagataagactaatcagggcatcccggaattgaatgcaagctccgtaggaatggccaaggcagctcttgaggcaattgatgaactggacctttttggagcccatggaggacgcaagtcagtgattcatgttctgccagatgaggtcgagcactgccagtctattctgttctccatgctgccaagagcgtcgacatctaaagaaattgatgctggacttctttccattatttccttcccggcctttgcagtggaagatgtaaaccttgtaaatgtgaccaaaaatgaaattatttctaagctccaggggcgttatggatgctgtcgcttccttcgagatggttataaaactccaagagaggaccctaatcgactgcattatgaccctgctgaactcaagctcttcgaaaacattgaatgtgagtggcctgtgttttggacatattttataatagatggagtcttcagtggtgatgctgttcaggtccaagaataccgagaggccctggagggaatactcatcagaggcaagaatgggatccgcctggtgcctgaactctacgctgtcccgcctaacaaggtagatgaagagtacaagaatcctcacacagtagaccgagttcctatggggaaggtgcctcatctgtggggccaatccttgtacatcctcagctcgctgttggcagagggattccttgccgctggtgaaatcgatcccttaaatagaagattttccacttcagtcaaacctgatgttgtagtacaagttactgttttggcagaaaacaatcacattaaggacttattgaggaaacacggggtgaacgtccagagtatcgcggacattcatccaattcaagtccagccgggccggattcttagtcacatatatgccaagcttggacggaataagaatatgaatttgagtgggcgaccgtatcgacatattggtgtccttggaacctctaaactatatgtgattaggaaccaaatctttacttttacaccccagcaaatgatggctcagacattcattctgctgtgctctccacaattagaaaac

SEQ ID NO: 4 - protein, 540 aa, Homo sapiens
MRSRSNSGVRLDGYARLVQQTILCYQNPVTGLLSASHEQKDAWVRDNIYSILAVWGLGMAYRKNADRDEDKAKAYELEQNVVKLMRGLLQCMMRQVAKVEKFKHTQSTKDSLHAKYNTATCGTVVGDDQWGHLQVDATSLFLLFLAQMTASGLRIIFTLDEVAFIQNLVFYIEAAYKVADYGMWERGDKTNQGIPELNASSVGMAKAALEAIDELDLFGAHGGRKSVIHVLPDEVEHCQSILFSMLPRASTSKEIDAGLLSIISFPAFAVEDVNLVNVTKNEIISKLQGRYGCCRFLRDGYKTPREDPNRLHYDPAELKLFENIECEWPVFWTYFIIDGVFSGDAVQVQEYREALEGILIRGKNGIRLVPELYAVPPNKVDEEYKNPHTVDRVPMGKVPHLWGQSLYILSSLLAEGFLAAGEIDPLNRRFSTSVKPDVVVQVTVLAENNHIKDLLRKHGVNVQSIADIHPIQVQPGRILSHIYAKLGRNKNMNLSGRPYRHIGVLGTSKLYVIRNQIFTFTPQQMMAQTFILLCSPQLEN

SEQ ID NO: 5 - DNA, 1779 bp, Homo sapiens
atgcggagcaggagcaattccggggtccgcttggacgggtacgcgcggctggtgcagcaaaccatcctgtgttaccagaatcccgtcacggggctgctgtcagccagccatgagcagaaggatgcctgggtgcgggataacatctacagtatcctggccgtgtggggcctgggcatggcctaccgtaagaatgcagaccgcgatgaggacaaggccaaggcctacgagctggagcagaacgtggtgaagctgatgcgaggtcttctccagtgcatgatgagacaggtggccaaagtggagaagttcaaacacactcagagcaccaaggacagcctgcacgccaagtacaacaccgccacctgtggcacggtggtgggcgacgaccagtggggccacctccaggtggatgccacctctctcttcctcctgttcctggcccagatgaccgcctcaggcttacgtatcattttcactctcgatgaggtggccttcatacagaatcttgtcttttacatagaagctgcatataaagtcgctgattatggaatgtgggagcgtggagataagactaatcagggcatcccggaattgaatgcaagctccgtaggaatggccaaggcagctcttgaggcaattgatgaactggacctttttggagcccatggaggacgcaagtcagtgattcatgttctgccagatgaggtcgagcactgccagtctattctgttctccatgctgccaagagcgtcgacatctaaagaaattgatgctggacttctttccattatttccttcccggcctttgcagtggaagatgtaaaccttgtaaatgtgaccaaaaatgaaattatttctaagctccaggggcgttatggatgctgtcgcttccttcgagatggttataaaactccaagagaggaccctaatcgactgcattatgaccctgctgaactcaagctcttcgaaaacattgaatgtgagtggcctgtgttttggacatattttataatagatggagtcttcagtggtgatgctgttcaggtccaagaataccgagaggccctggagggaatactcatcagaggcaagaatgggatccgcctggtgcctgaactctacgctgtcccgcctaacaaggtagatgaagagtacaagaatcctcacacagtagaccgagttcctatggggaaggtgcctcatctgtggggccaatccttgtacatcctcagctcgctgttggcagagggattccttgccgctggtgaaatcgatcccttaaatagaagattttccacttcagtcaaacctgatgttgtagtacaagttactgttttggcagaaaacaatcacattaaggacttattgaggaaacacggggtgaacgtccagagtatcgcggacattcatccaattcaagtccagccgggccggattcttagtcacatatatgccaagcttggacggaataagaatatgaatttgagtgggcgaccgtatcgacatattggtgtccttggaacctctaaactatatgtgattaggaaccaaatctttacttttacaccccagttcaccgaccagcatcacttctacctggccctcgacaatgagatgatcgtggagatgctaaggatcgagctggcctacctgtgcacctgctggaggatgacgggcagacccacactcaccttccccatcagtcgcaccatgctcagtaactccagggatttcagcaggctccctcctgccccaggggtggtttgggctcttacaaatgtt

SEQ ID NO: 6 - protein, 593 aa, Homo sapiens [sequence omitted]

SEQ ID NO: 7 - DNA, 2073 bp, Homo sapiens
atggtgttgatgaaaatgtttcagtgcataacggaaaaaaagtctgcccagactaaactaattcatcactttttttccttagcaaatgatggctcagacattcattctgctgtgctctccacaattagaaaactagaggatggatattttggaggagccagagtaaaattagggaacctttcggaatttctcaccacatcgttctacacatatctgacttttctggatccagactgtgatgagaagttgtttgacaatgccagcgaagggactttcagtcctgatagtgattcagatttggtaggatatctggaagacacctgtaatcaagaaagccaagacgaacttgaccattatatcaaccaccttctgcaaagcacatcgttgaggtcctatctgcctcctctttgtaagaacacagaagaccgccatgtcttcagtgctatccactccacgcgggacatactttctgtgatggcaaaagcaaagggtttggaagttccatttgttcccatgactttgccgactaaagttctaagtgcccaccgtaaatcactgaatcttgttgattctcctcagccactcctagaaaaggttcctgaaagtgactttcagtggcccagagatgaccatggtgacgtggactgtgagaagctggttgagcagctaaaagattgttcgaacctacaggaccaagcagacattctgtacattctttatgtcataaagggtcccagctgggacacaaatctctctggacagcacggggtcaccgttcaaaaccttcttggtgagctctatgggaaagccggcttgaaccaggagtggggtctgattcgctacatctcaggccttctcaggaagaaagtggaggtcctggctgaggcctgcacagacctgctttcgcaccagaagcagctcaccgtgggcctgccgcccgagccccgggagaagatcatctctgcgccccttcccccagaggagctcacaaaactcatctacgaggccagtgggcaggacatcagcattgccgtcctcacgcaggagattgtggtttacctggccatgtatgtcagggcgcagcccagcctctttgtggagatgctgagactccggattggactgatcattcaggtgatggccacggagctggcacggagcctgaactgctcaggagaagaggcttctgaaagtttgatgaacctcagccctttcgatatgaaaaatctcctgcaccatattctaagtgggaaagagtttggcgttgaaagaagtgtgcgccctatccactcctccacatccagccctaccatctccatccacgaggtgggccataccggagtcaccaaaactgagaggagtggcattaacagactgaggagtgaaatgaaacagatgactaggcggtttagtgctgatgaacagttcttttctgtgggccaggccgcgtccagcagtgcgcattcctccaagtctgcgaggtccagcaccccatcctcgcccactggcacgtcatcctcagactcgggaggacatcacatcggctggggtgagcggcagggccagtggctgcgcaggagaaggctggatggggccatcaacagggtccccgtgggattctaccagagggtgtggaagatcctccagaagtgccacggtctctccatcgatggttatgtcctcccatcctcgacgacccgagagatgaccccgcatgagatcaagtttgctgtccatgtcgaatcggtgctgaaccgcgtgccgcagcccgagtaccggcagctgctggtggaagccatcatggtgctgacgctgctctcggacacggagatgaccagcatcgggggcatcatccacgtggaccagatcgtgcagatggccagtcagctgttcttgcaggaccaggtgtcaattggtgccatggacaccctggagaaagaccaagccacaggaatctgccacttcttttatgacagcgctccgagtggggcttatgggacgatgacctacctaacaagagcagtggcttcttatttgcaggaattgttgcccaattcgggctgccagatgcaa

SEQ ID NO: 8 - protein, 691 aa, Homo sapiens [sequence omitted]

SEQ ID NO: 9 - DNA, 3606 bp, Homo sapiens
atgcggagcaggagcaattccggggtccgcttggacgggtacgcgcggctggtgcagcaaaccatcctgtgttaccagaatcccgtcacggggctgctgtcagccagccatgagcagaaggatgcctgggtgcgggataacatctacagtatcctggccgtgtggggcctgggcatggcctaccgtaagaatgcagaccgcgatgaggacaaggccaaggcctacgagctggagcagaacgtggtgaagctgatgcgaggtcttctccagtgcatgatgagacaggtggccaaagtggagaagttcaaacacactcagagcaccaaggacagcctgcacgccaagtacaacaccgccacctgtggcacggtggtgggcgacgaccagtggggccacctccaggtggatgccacctctctcttcctcctgttcctggcccagatgaccgcctcaggcttacgtatcattttcactctcgatgaggtggccttcatacagaatcttgtcttttacatagaagctgcatataaagtcgctgattatggaatgtgggagcgtggagataagactaatcagggcatcccggaattgaatgcaagctccgtaggaatggccaagtctattctgttctccatgctgccaagagcgtcgacatctaaagaaattgatgctggacttctttccattatttccttcccggcctttgcagtggaagatgtaaaccttgtaaatgtgaccaaaaatgaaattatttctaagctccaggggcgttatggatgctgtcgcttccttcgagatggttataaaactccaagagaggaccctaatcgactgcattatgaccctgctgaactcaagctcttcgaaaacattgaatgtgagtggcctgtgttttggacatattttataatagatggagtcttcagtggtgatgctgttcaggtccaagaataccgagaggccctggagggaatactcatcagaggcaagaatgggatccgcctggtgcctgaactctacgctgtcccgcctaacaaggtagatgaagagtacaagaatcctcacacagtagaccgagttcctatggggaaggtgcctcatctgtggggccaatccttgtacatcctcagctcgctgttggcagagggattccttgccgctggtgaaatcgatcccttaaatagaagattttccacttcagtcaaacctgatgttgtagtacaagttactgttttggcagaaaacaatcacattaaggacttattgaggaaacacggggtgaacgtccagagtatcgcggacattcatccaattcaagtccagccgggccggattcttagtcacatatatgccaagcttggacggaataagaatatgaatttgagtgggcgaccgtatcgacatattggtgtccttggaacctctaaactatatgtgattaggaaccaaatctttacttttacaccccagttcaccgaccagcatcacttctacctggccctcgacaatgagatgatcgtggagatgctaaggatcgagctggcctacctgtgcacctgctggaggatgacgggcagacccacactcaccttccccatcagtcgcaccatgctcacaaatgatggctcagacattcattctgctgtgctctccacaattagaaaactagaggatggatattttggaggagccagagtaaaattagggaacctttcggaatttctcaccacatcgttctacacatatctgacttttctggatccagactgtgatgagaagttgtttgacaatgccagcgaagggactttcagtcctgatagtgattcagatttggtaggatatctggaagacacctgtaatcaagaaagccaagacgaacttgaccattatatcaaccaccttctgcaaagcacatcgttgaggtcctatctgcctcctctttgtaagaacacagaagaccgccatgtcttcagtgctatccactccacgcgggacatactttctgtgatggcaaaagcaaagggtttggaagttccatttgttcccatgactttgccgactaaagttctaagtgcccaccgtaaatcactgaatcttgttgattctcctcagccactcctagaaaaggttcctgaaagtgactttcagtggcccagagatgaccatggtgacgtggactgtgagaagctggttgagcagctaaaagattgttcgaacctacaggaccaagcagacattctgtacattctttatgtcataaagggtcccagctgggacacaaatctctctggacagcacggggtcaccgttcaaaaccttcttggtgagctctatgggaaagccggcttgaaccaggagtggggtctgattcgctacatctcaggccttctcaggaagaaagtggaggtcctggctgaggcctgcacagacctgctttcgcaccagaagcagctcaccgtgggcctgccgcccgagccccgggagaagatcatctctgcgccccttcccccagaggagctcacaaaactcatctacgaggccagtgggcaggacatcagcattgccgtcctcacgcaggagattgtggtttacctggccatgtatgtcagggcgcagcccagcctctttgtggagatgctgagactccggattggactgatcattcaggtgatggccacggagctggcacggagcctgaactgctcaggagaagaggcttctgaaagtttgatgaacctcagccctttcgatatgaaaaatctcctgcaccatattctaagtgggaaagagtttggcgttgaaagaagtgtgcgccctatccactcctccacatccagccctaccatctccatccacgaggtgggccataccggagtcaccaaaactgagaggagtggcattaacagactgaggagtgaaatgaaacagatgactaggcggtttagtgctgatgaacagttcttttctgtgggccaggccgcgtccagcagtgcgcattcctccaagtctgcgaggtccagcaccccatcctcgcccactggcacgtcatcctcagactcgggaggacatcacatcggctggggtgagcggcagggccagtggctgcgcaggagaaggctggatggggccatcaacagggtccccgtgggattctaccagagggtgtggaagatcctccagaagtgccacggtctctccatcgatggttatgtcctcccatcctcgacgacccgagagatgaccccgcatgagatcaagtttgctgtccatgtcgaatcggtgctgaaccgcgtgccgcagcccgagtaccggcagctgctggtggaagccatcatggtgctgacgctgctctcggacacggagatgaccagcatcgggggcatcatccacgtggaccagatcgtgcagatggccagtcagctgttcttgcaggaccaggtgtcaattggtgccatggacaccctggagaaagaccaagccacaggaatctgccacttcttttatgacagcgctccgagtggggcttatgggacgatgacctacctaacaagagcagtggcttcttatttgcaggaattgttgcccaattcgggctgccagatgcaa

SEQ ID NO: 10 - protein, 1202 aa, Homo sapiens
MRSRSNSGVRLDGYARLVQQTILCYQNPVTGLLSASHEQKDAWVRDNIYSILAVWGLGMAYRKNADRDEDKAKAYELEQNVVKLMRGLLQCMMRQVAKVEKFKHTQSTKDSLHAKYNTATCGTVVGDDQWGHLQVDATSLFLLFLAQMTASGLRIIFTLDEVAFIQNLVFYIEAAYKVADYGMWERGDKTNQGIPELNASSVGMAKSILFSMLPRASTSKEIDAGLLSIISFPAFAVEDVNLVNVTKNEIISKLQGRYGCCRFLRDGYKTPREDPNRLHYDPAELKLFENIECEWPVFWTYFIIDGVFSGDAVQVQEYREALEGILIRGKNGIRLVPELYAVPPNKVDEEYKNPHTVDRVPMGKVPHLWGQSLYILSSLLAEGFLAAGEIDPLNRRFSTSVKPDVVVQVTVLAENNHIKDLLRKHGVNVQSIADIHPIQVQPGRILSHIYAKLGRNKNMNLSGRPYRHIGVLGTSKLYVIRNQIFTFTPQFTDQHHFYLALDNEMIVEMLRIELAYLCTCWRMTGRPTLTFPISRTMLTNDGSDIHSAVLSTIRKLEDGYFGGARVKLGNLSEFLTTSFYTYLTFLDPDCDEKLFDNASEGTFSPDSDSDLVGYLEDTCNQESQDELDHYINHLLQSTSLRSYLPPLCKNTEDRHVFSAIHSTRDILSVMAKAKGLEVPFVPMTLPTKVLSAHRKSLNLVDSPQPLLEKVPESDFQWPRDDHGDVDCEKLVEQLKDCSNLQDQADILYILYVIKGPSWDTNLSGQHGVTVQNLLGELYGKAGLNQEWGLIRYISGLLRKKVEVLAEACTDLLSHQKQLTVGLPPEPREKIISAPLPPEELTKLIYEASGQDISIAVLTQEIVVYLAMYVRAQPSLFVEMLRLRIGLIIQVMATELARSLNCSGEEASESLMNLSPFDMKNLLHHILSGKEFGVERSVRPIHSSTSSPTISIHEVGHTGVTKTERSGINRLRSEMKQMTRRFSADEQFFSVGQAASSSAHSSKSARSSTPSSPTGTSSSDSGGHHIGWGERQGQWLRRRRLDGAINRVPVGFYQRVWKILQKCHGLSIDGYVLPSSTTREMTPHEIKFAVHVESVLNRVPQPEYRQLLVEAIMVLTLLSDTEMTSIGGIIHVDQIVQMASQLFLQDQVSIGAMDTLEKDQATGICHFFYDSAPSGAYGTMTYLTRAVASYLQELLPNSGCQMQ

SEQ ID NO: 11 - DNA, 20 bp, Homo sapiens
gaaagaagtgaggtccagca

SEQ ID NO: 12 - DNA, 20 bp, Homo sapiens
tacaccccagcaaatgatgg

SEQ ID NO: 13 - DNA, 20 bp, Homo sapiens
accatgctcagtaactccag

SEQ ID NO: 14 - DNA, 20 bp, Homo sapiens
ttttccttagcaaatgatgg

SEQ ID NO: 15 - DNA, 20 bp, Homo sapiens
atggtgttgatgaaaatgtt

SEQ ID NO: 16 - DNA, 20 bp, Homo sapiens
aatggccaagtctattctgt

SEQ ID NO: 17 - protein, 10 aa, Homo sapiens
GVERSEVQHP

SEQ ID NO: 18 - protein, 10 aa, Homo sapiens
TFTPQQMMAQ

SEQ ID NO: 19 - protein, 10 aa, Homo sapiens
SRTMLSNSRD

SEQ ID NO: 20 - protein, 10 aa, Homo sapiens
FFSLANDGSG

SEQ ID NO: 21 - protein, 10 aa, Homo sapiens
VGMAKSILFS

SEQ ID NO: 22 - DNA, 28 bp, Homo sapiens
gaaagtttgatgaacctcagccctttcg

SEQ ID NO: 23 - DNA, 28 bp, Homo sapiens
aaacttgatctcatgcggggtcatctct

SEQ ID NO: 24 - DNA, 28 bp, Homo sapiens
agagtatcgcggacattcatccaattca

SEQ ID NO: 25 - DNA, 27 bp, Homo sapiens
acgatgtggtgagaaattccgaaaggt

SEQ ID NO: 26 - DNA, 28 bp, Homo sapiens
ataacatctacagtatcctggccgtgtg

SEQ ID NO: 27 - DNA, 28 bp, Homo sapiens
tgaacagcatcaccactgaagactccat

SEQ ID NO: 28 - DNA, 27 bp, Homo sapiens
atgcggagcaggagcaattccggggtc

SEQ ID NO: 29 - DNA, 28 bp, Homo sapiens
ccccagccgatgtgatgtcctcccgagt

SEQ ID NO: 30 - DNA, 28 bp, Homo sapiens
gttttctaattgtggagagcacagcaga

SEQ ID NO: 31 - DNA, 28 bp, Homo sapiens
aacatttgtaagagcccaaaccacccct

SEQ ID NO: 32 - DNA, 28 bp, Homo sapiens
atggtgttgatgaaaatgtttcagtgca

SEQ ID NO: 33 - DNA, 28 bp, Homo sapiens
ttgcatctggcagcccgaattgggcaac

SEQ ID NO: 34 - DNA, 28 bp, Homo sapiens
ttgcatctggcagcccgaattgggcaac

(19) United States
(12) Patent Application Publication
(10) Pub. No.: US 2006/0127913 A1
(43) Pub. Date: Jun. 15, 2006

(54) Alternatively spliced isoforms of human PHKA2

(76) Inventors: Jason M. Johnson, Seattle, WA (US); Philip W. Garrett-Engele, Seattle, WA (US); Christopher D. Armour, Kirkland, WA (US); John C. Castle, Seattle, WA (US)

Correspondence Address: R. Douglas Bradley;Merck & Co., Inc., Patent Department RY60-30, P.O. Box 2000, Rahway, NJ, 07065-0907, US

(21) Appl. No.: 10/648,139

(22) Filed: Aug. 26, 2003

Related U.S. Application Data
(60) Provisional application No. 60/408,058, filed on Sep. 3, 2002.
(60) Provisional application No. 60/431,474, filed on Dec. 5, 2002.

(51) Int. Cl. C12Q 1/68; C07H 21/04; C12P 21/06; C12N 9/12

(52) U.S. Cl. 435/6; 435/69.1; 435/194; 435/320.1; 435/325; 536/23.2

(57) ABSTRACT

The present invention features nucleic acids and polypeptides encoding four novel splice variant isoforms of PHKA2. The polynucleotide sequences of PHKA2sv3, PHKA2sv4, PHKA2sv6 and PHKA2sv7 are provided by SEQ ID NO 1, SEQ ID NO 3, SEQ ID NO 5 and SEQ ID NO 6, respectively. The amino acid sequences for PHKA2sv3, PHKA2sv4, and PHKA2sv7 are provided by SEQ ID NO 2, SEQ ID NO 4, and SEQ ID NO 7, respectively. The present invention also provides methods for using PHKA2sv3, PHKA2sv4, and PHKA2sv7 polynucleotides and proteins to screen for compounds that bind to PHKA2sv3, PHKA2sv4, and PHKA2sv7, respectively.

This application claims priority to U.S. Provisional Patent Application Ser. No. 06/408,058 filed on Sep. 3, 2002, and U.S. Provisional Patent Application Ser. No. 60/431,474 filed on Dec. 05, 2002, each of which is incorporated by reference herein in its entirety.

### BACKGROUND OF THE INVENTION

The references cited herein are not admitted to be prior art to the claimed invention.

Glycogen is the major storage form of glucose. Excess glucose obtained from amino acids and lactate through the gluconeogenesis pathway and from the diet is converted to glycogen. Glycogen is then stored for future use primarily in the liver and skeletal muscles. Mobilization of stored glycogen occurs through the process of glycogenolysis, in which single glucose-1-phosphate molecules are cleaved from glycogen. The resulting glucose molecules are released into the blood stream for utilization predominantly by brain and muscle cells. Thus, glycogenolysis is crucial for maintaining blood glucose levels during periods of exercise, sleep, and fasting.

Phosphorylase kinase (PHK) is a key enzyme in the control of glycogen metabolism. PHK catalyzes the conversion of inactive glycogen phosphorylase b to its active form glycogen phosphorylase a, which results in the breakdown of glycogen.

PHK is one of the most complex kinases identified. It is comprised of four different subunits arranged as a (αβγδ)₄ tetramer. The α, β and δ subunits are important for the regulation of PHK activity, while the γ subunit is the catalytic subunit of the enzyme. The three regulatory subunits inhibit the phosphotransferase activity of the γ subunit. Cyclic AMP-dependent kinase (cAMPK) phosphorylates the α and β subunits in response to adrenaline, which relieves the inhibition of the γ subunit and activates PHK (Brushia and Walsh, 1999 Front. Biosci., 4:618-641). In addition, the α and β subunits can be autophosphorylated by PHK itself on at least three serine residues. The δ subunit, also known as calmodulin, receives intracellular Ca²⁺ signals and enhances the activity of the enzyme by relieving PHK inhibition via the γ subunit (Hendrickx and Willems, 1996 Hum. Genet., 97:551-556). The γ subunit contains a kinase domain, an autoinhibitory domain and a calmodulin binding domain (Dasgupta and Blumenthal, 1989 J. Biol. Chem., 264:17156-17163). There are several isoforms of each of these PHK subunits. While some of these isoforms are encoded by different genes, others result from differential splicing of the same gene (Hendrickx and Willems, 1996 Hum. Genet., 97:551-556).

The liver isoform of the human α subunit is encoded by the PHKA2 gene (Hirono, et al., 1995 Biochem. Mol. Biol. Int., 36:505-511). The PHKA2 reference gene (NM_{—}000292) consists of 33 exons, spanning over 65 kilobases (Hendrickx et al., 1999 Am. J. Hum. Genet., 64:1541-1549). PHKA2 protein encoded by the PHKA2 gene shares 68% amino acid homology with the muscle isoform of the human a subunit (encoded by PHKA1) and 93% homology with the rabbit muscle PHK α subunit (Hirono et al., 1995 Biochem. Mol. Biol. Int., 36:505-511). In addition, three splice variants of PHKA2, one missing exon 4, one missing exon 29, and one missing exons 28 and 29, have previously been described (Hirono et al., 1995 Biochem. Mol. Biol. Int., 36:505-511; Wullrich et al., 1993 J. Biol. Chem. 268:23208-23214).

Mutations in PHKA2 result in the most common glycogen-storage disease, X-linked glycogenesis (XLG) (Hendrickx et al., 1999 Am. J. Hum. Genet., 64:1541-1549). Patients with XLG are unable to breakdown glycogen, and thus develop enlarged livers and experience growth retardation (Willems et al., 1990 Eur. J. Pediatr., 149:268-271). Although patients are able to store excess glucose as glycogen, they are unable to breakdown the glycogen into glucose. Therefore at times when extra glucose is required, patients often suffer from hypoglycemia. Currently there are no treatments for glycogen storage diseases except for dietary therapies.

Hendrickx et al. compiled a list of 30 different mutations in PHKA2 that result in XLG (1999 Am. J. Hum. Genet., 64:1541-1549). Of these mutations, thirteen are missense mutations that result in a single amino acid change, five are nonsense mutations that result in a premature stop codon, eleven are either insertions or deletions, and one results in the elimination of a splice site that results in an exon skipping event. These mutations result in either PHK activity deficiency in the liver, leukocytes and erythrocytes (XLG I), or normal PHK activity in leukocytes and erythrocytes but varying activity in the liver (XLG II) (Hendrickx et al., 1994 Genomics, 21:620-625).

Curcumin (diferuloylmethane), the major active compound in tumeric, had been demonstrated to be a non-competitive inhibitor of phosphorylase kinase (Reddy and Aggarwal, 1994 FEBS Letters, 341:19-22). The authors hypothesize that curcumin interacts with the β subunit. However, curcumin also significantly inhibits pp60^{c-src} tyrosine kinase, protein kinase C, and protein kinase A at slightly higher concentrations. Curcumin's activity as an inhibitor of PHK has been used as a treatment for psoriasis (US Patent Application Number: 20010051184). In addition, the PHK inhibitor anthralin has also been described as a treatment for psoriasis (U.S. Pat. No. 5,925,376). Thus, while some compounds have been identified that alter PHK activity to achieve a therapeutic benefit, there still remains a substantial need in the art for additional compounds that specifically inhibit phosphorylase kinase activity.

Furthermore, due to its role in blood glucose homeostasis, PHKs may also play a significant role in other metabolic disorders, including, for example diabetes and obesity. Because of the importance of PHKA2 as a drug target and its roles in metabolism and disease, there is a need in the art for PHKA2 polynucleotides and proteins and methods of use thereof that can be used to identify compounds that selectively bind to isoforms of human PHKA2. The present invention is directed towards novel PHKA2 isoforms and uses thereof.

### SUMMARY OF THE INVENTION

Microarray experiments and RT-PCR have been used to identify and confirm the presence of four novel splice variants of human PHKA2 mRNA. More specifically, the present invention features polynucleotides encoding different protein isoforms of PHKA2. A polynucleotide sequence encoding PHKA2sv3 is provided by SEQ ID NO 1. An amino acid sequence for PHKA2sv3 is provided by SEQ ID NO 2. A polynucleotide sequence encoding PHKA2sv4 is provided by SEQ ID NO 3. An amino acid sequence for PHKA2sv4 is provided by SEQ ID NO 4. A polynucleotide sequence encoding PHKA2sv6.1 is provided by SEQ ID NO 5. An amino acid sequence for PHKA2sv6.1 is provided by SEQ ID NO 6. A polynucleotide sequence encoding PHKA2sv6.2 is provided by SEQ ID NO 7. An amino acid sequence for PHKA2sv6.2 is provided by SEQ ID NO 8. A polynucleotide sequence encoding PHKA2sv7 is provided by SEQ ID NO 9. An amino acid sequence for PHKA2sv7 is provided by SEQ ID NO 10.

Thus, a first aspect of the present invention describes a purified PHKA2sv3 encoding nucleic acid, a purified PHKA2sv4 encoding nucleic acid, a purified PHKA2sv6.1, a purified PHKA2sv6.2 encoding nucleic acid and a purified PHKA2sv7 encoding nucleic acid. The PHKA2sv3 encoding nucleic acid comprises SEQ ID NO 1 or the complement thereof. The PHKA2sv4 encoding nucleic acid comprises SEQ ID NO 3 or the complement thereof. The PHKA2sv6.1 encoding nucleic acid comprises SEQ ID NO 5 or the complement thereof. The PHKA2sv6.2 encoding nucleic acid comprises SEQ ID NO 7 or the complement thereof. The PHKA2sv7 encoding nucleic acid comprises SEQ ID NO 9 or the complement thereof. Reference to the presence of one region does not indicate that another region is not present. For example, in different embodiments the inventive nucleic acid can comprise, consist, or consist essentially of a nucleic acid encoding for SEQ ID NO 1, can comprise, consist, or consist essentially of the nucleic acid sequence of SEQ ID NO 3, can comprise, consist, or consist essentially of the nucleic acid sequence of SEQ ID NO 5, can comprise, consist, or consist essentially of the nucleic acid sequence of SEQ ID NO 7, or alternatively, can comprise, consist, or consist essentially of the nucleic acid sequence of SEQ ID NO 9.

Another aspect of the present invention describes a purified PHKA2sv3 polypeptide that can comprise, consist or consist essentially of the amino acid sequence of SEQ ID NO 2. An additional aspect describes a purified PHKA2sv4 polypeptide that can comprise, consist, or consist essentially of the amino acid sequence of SEQ ID NO 4. An additional aspect describes a purified PHKA2sv6.1 polypeptide that can comprise, consist, or consist essentially of the amino acid sequence of SEQ ID NO 6. An additional aspect describes a purified PHKA2sv6.2 polypeptide that can comprise, consist, or consist essentially of the amino acid sequence of SEQ ID NO 8. An additional aspect describes a purified PHKA2sv7 polypeptide that can comprise, consist, or consist essentially of the amino acid sequence of SEQ ID NO 10.

Another aspect of the present invention describes expression vectors. In one embodiment of the invention, the inventive expression vector comprises a nucleotide sequence encoding a polypeptide comprising, consisting, or consisting essentially of SEQ ID NO 2, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 4, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 6, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 8, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 10, wherein the nucleotide sequence is transcriptionally coupled to an exogenous promoter.

Alternatively, the nucleotide sequence comprises, consists, or consists essentially of SEQ ID NO 1, and is transcriptionally coupled to an exogenous promoter. In another embodiment, the nucleotide sequence comprises, consists, or consists essentially of SEQ ID NO 3, and is transcriptionally coupled to an exogenous promoter. In another embodiment, the nucleotide sequence comprises, consists, or consists essentially of SEQ ID NO 5, and is transcriptionally coupled to an exogenous promoter. In another embodiment, the nucleotide sequence comprises, consists, or consists essentially of SEQ ID NO 7, and is transcriptionally coupled to an exogenous promoter. In another embodiment, the nucleotide sequence comprises, consists, or consists essentially of SEQ ID NO 9, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention, describes a recombinant cell made by a process comprising the step of introducing into the cell an expression vector comprising a nucleotide sequence comprising, consisting, or consisting essentially of SEQ ID NO 1, SEQ ID NO 3, SEQ ID NO 5, SEQ ID NO 7, SEQ ID NO 9 or a nucleotide sequence encoding a polypeptide comprising, consisting, or consisting essentially of an amino acid sequence of SEQ ID NO 2, SEQ ID NO 4, SEQ ID NO 6, SEQ ID NO 8 or SEQ ID NO 10 wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing PHKA2sv3, PHKA2sv4, PHKA2sv6.1, PHKA2sv6.2 or PHKA2sv7 polypeptide comprising SEQ ID NO 2, SEQ ID NO 4, SEQ ID NO 6, SEQ ID NO 8 or SEQ ID NO 10, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to PHKA2sv3 as compared to one or more PHKA2 isoform polypeptides that are not PHKA2sv3. In another embodiment, a purified antibody preparation is provided comprising antibody that binds selectively to PHKA2sv4 as compared to PHKA2 isoform polypeptide that is not PHKA2sv4. In another embodiment, a purified antibody preparation is provided comprising antibody that binds selectively to PHKA2sv6.1 as compared to PHKA2 isoform polypeptide that is not PHKA2sv6.1. In another embodiment, a purified antibody preparation is provided comprising antibody that binds selectively to PHKA2sv6.2 as compared to PHKA2 isoform polypeptide that is not PHKA2sv6.2. In another embodiment, a purified antibody preparation is provided comprising antibody that binds selectively to PHKA2sv7 as compared to PHKA2 isoform polypeptide that is not PHKA2sv7.

Another aspect of the present invention provides a method of screening for a compound that binds to PHKA2sv3, PHKA2sv4, PHKA2sv6.1, PHKA2sv6.2, or PHKA2sv7, or fragments thereof. In one embodiment, the method comprises the steps of: (a) expressing a polypeptide comprising the amino acid sequence of SEQ ID NO 2 or a fragment thereof from recombinant nucleic acid; (b) providing to said polypeptide a labeled PHKA2 ligand that binds to said polypeptide and a test preparation comprising one or more test compounds; (c) and measuring the effect of said test preparation on binding of said test preparation to said polypeptide comprising SEQ ID NO 2. Alternatively, this method could be performed using SEQ ID NO 4, SEQ ID NO 6, SEQ ID NO 8, or SEQ ID NO 10, in place of SEQ ID NO 2.

In another embodiment of the method, a compound is identified that binds selectively to PHKA2sv3 polypeptide as compared to one or more PHKA2 isoform polypeptides that are not PHKA2sv3. This method comprises the steps of: providing a PHKA2sv3 polypeptide comprising SEQ ID NO 2; providing a PHKA2 isoform polypeptide that is not PHKA2sv3, contacting said PHKA2sv3 polypeptide and said PHKA2 isoform polypeptide that is not PHKA2sv3 with a test preparation comprising one or more test compounds; and determining the binding of said test preparation to said PHKA2sv3 polypeptide and to PHKA2 isoform polypeptide that is not PHKA2sv3, wherein a compound which binds to said PHKA2sv3 polypeptide but does not bind to said PHKA2 isoform polypeptide that is not PHKA2sv3 is a compound that selectively binds said PHKA2sv3 polypeptide. Alternatively, the same method can be performed using PHKA2sv4 polypeptide comprising, consisting, or consisting essentially of SEQ ID NO 4. Alternatively, the same method can be performed using PHKA2sv6.1 polypeptide comprising, consisting, or consisting essentially of SEQ ID NO 6. Alternatively, the same method can be performed using PHKA2sv6.2 polypeptide comprising, consisting, or consisting essentially of SEQ ID NO 8. Alternatively, the same method can be performed using PHKA2sv7 polypeptide comprising, consisting, or consisting essentially of SEQ ID NO 10.

In another embodiment of the invention, a method is provided for screening for a compound able to bind to or interact with a PHKA2sv3 protein or a fragment thereof comprising the steps of: expressing a PHKA2sv3 polypeptide comprising SEQ ID NO 2 or a fragment thereof from a recombinant nucleic acid; providing to said polypeptide a labeled PHKA2 ligand that binds to said polypeptide and a test preparation comprising one or more compounds; and measuring the effect of said test preparation on binding of said labeled PHKA2 ligand to said polypeptide, wherein a test preparation that alters the binding of said labeled PHKA2 ligand to said polypeptide contains a compound that binds to or interacts with said polypeptide. In an alternative embodiment, the method is performed using PHKA2sv4 polypeptide comprising, consisting, or consisting essentially of SEQ ID NO 4 or a fragment thereof. In an alternative embodiment, the method is performed using PHKA2sv6.1 polypeptide comprising, consisting, or consisting essentially of SEQ ID NO 6 or a fragment thereof. In an alternative embodiment, the method is performed using PHKA2sv6.2 polypeptide comprising, consisting, or consisting essentially of SEQ ID NO 8 or a fragment thereof. In an alternative embodiment, the method is performed using PHKA2sv7 polypeptide comprising, consisting, or consisting essentially of SEQ ID NO 10 or a fragment thereof.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1A illustrates the exon structure of PHKA2 mRNA corresponding to the known reference form of PHKA2 mRNA (labeled NM_{—}000292). FIG. 1B illustrates one of the inventive short form splice variants of PHKA2 mRNA (labeled PHKA2sv3). The small arrows above exons 26 and 32 show the positions of the oligonucleotide primers used to perform RT-PCR assays to confirm the exon structure of PHKA2 mRNA in 44 human samples (see Table 1). The nucleotide sequences shown in boxes below the exon structure diagrams of the PHKA2 and PHKA2sv3 mRNAs depict the nucleotides sequences of the exon junctions resulting from the splicing of exon 26 to exon 27, and exon 29 to exon 30 in the case of the PHKA2 mRNA (FIG. 1A); and the splicing of exon 26 to exon 30 in the case of PHKA2sv3 mRNA (FIG. 1B). In FIG. 1A, the nucleotides shown in italics represent the 20 nucleotides at the 3′ end of exon 26 and the nucleotides shown in underline represent the 20 nucleotides at the 5′ end of exon 30. In FIG. 1B, nucleotides in italics associated with the exon 26 to exon 30 splice junction represent the 20 nucleotides at the 3′ end of exon 26, while the nucleotides in underline associated with the exon 26 to exon 30 splice junction represent the 20 nucleotides at the 5′ end of exon 30.

FIG. 2A illustrates the exon structure of PHKA2 mRNA corresponding to the known reference form of PHKA2 mRNA (labeled NM_{—}000292). FIG. 2B illustrates one of the inventive short form splice variants of PHKA2 mRNA (labeled PHKA2sv4). The small arrows above exons 14 and 18 show the positions of the oligonucleotide primers used to perform RT-PCR assays to confirm the exon structure of PHKA2 mRNA in 44 human samples (see Table 1). The nucleotide sequences shown in boxes below the exon structure diagrams of the PHKA2 and PHKA2sv4 mRNAs depict the nucleotides sequences of the exon junctions resulting from the splicing of exon 15 to exon 16, and exon 16 to exon 17 in the case of the PHKA2 mRNA (FIG. 2A); and the splicing of exon 15 to exon 17 in the case of PHKA2sv4 mRNA (FIG. 2B). In FIG. 2A, the nucleotides shown in italics represent the 20 nucleotides at the 3′ end of exon 15 and the nucleotides shown in underline represent the 20 nucleotides at the 5′ end of exon 17. In FIG. 2B, the nucleotides in italics associated with the exon 15 to exon 17 splice junction represent the 20 nucleotides at the 3′ end of exon 15, while the nucleotides in underline associated with the exon 15 to exon 17 splice junction represent the 20 nucleotides at the 5′ end of exon 17.

FIG. 3A illustrates the exon structure of PHKA2 mRNA corresponding to the known reference form of PHKA2 mRNA (labeled NM_{—}000292). FIG. 3B illustrates one of the inventive splice variants of PHKA2 mRNA (labeled PHKA2sv6). The small arrows above exons 14 and 18 show the positions of the oligonucleotide primers used to perform RT-PCR assays to confirm the exon structure of PHKA2 mRNA in 44 human samples (see Table 1). The nucleotide sequences shown in boxes below the exon structure diagrams of the PHKA2 and PHKA2sv6 mRNAs depict the nucleotides sequences of the exon junctions resulting from the splicing of exon 16 to exon 17 in the case of the PHKA2 mRNA (FIG. 3A); and the junctions of exon 16 to intron 16 and intron 16 to exon 17 in the case of PHKA2sv6 mRNA (FIG. 3B). In FIG. 3A, the nucleotides shown in bold represent the 20 nucleotides at the 3′ end of exon 16, while the nucleotides shown in underline represent the 20 nucleotides at the 5′ end of exon 17. In FIG. 3B, the nucleotides in italics associated with the exon 16 to intron 16 junction represent the 20 nucleotides at the 3′ end of exon 16, while the nucleotides in underline associated with the exon 16 to intron 16 junction represent the 20 nucleotides at the 5′ end of intron 16. In addition, the nucleotides in italics associated with the intron 16 to exon 17 junction represent the 20 nucleotides at the 3′ end of intron 16, while the nucleotides in underline associated with the intron 16 to exon 17 junction represent the 20 nucleotides at the 5′ end of exon 17.

FIG. 4A illustrates the exon structure of PHKA2 mRNA corresponding to the known reference form of PHKA2 mRNA (labeled NM_{—}000292). FIG. 4B illustrates one of the inventive short form splice variants of PHKA2 mRNA (labeled PHKA2sv7). The small arrows above exons 2 and 11 show the positions of the oligonucleotide primers used to perform RT-PCR assays to confirm the exon structure of PHKA2 mRNA in 44 human samples (see Table 1). The nucleotide sequences shown in boxes below the exon structure diagrams of the PHKA2 and PHKA2sv7 mRNAs depict the nucleotides sequences of the exon junctions resulting from the splicing of exon 6 to exon 7, and exon 7 to exon 8 in the case of the PHKA2 mRNA (FIG. 4A); and the splicing of exon 6 to exon 8 in the case of PHKA2sv7 mRNA (FIG. 4B). In FIG. 4A, the nucleotides shown in italics represent the 20 nucleotides at the 3′ end of exon 6 and the nucleotides shown in underline represent the 20 nucleotides at the 5′ end of exon 8. In FIG. 4B, the nucleotides in italics associated with the exon 6 to exon 8 splice junction represent the 20 nucleotides at the 3′ end of exon 6, while the nucleotides in underline associated with the exon 6 to exon 8 splice junction represent the 20 nucleotides at the 5′ end of exon 8.

### DEFINITIONS

Unless defined otherwise, all technical and scientific terms used herein have the meaning commonly understood by one of ordinary skill in the art to which this invention belongs.

As used herein, “PHKA2” refers to a liver isoform of a human phosphorylase kinase alpha subunit protein (NP_{—}000283). In contrast, reference to a PHKA2 isoform, includes NP_{—}000283 and other polypeptide isoform variants of PHKA2.

As used herein, “PHKA2” refers to polynucleotides encoding PHKA2.

As used herein, “PHKA2sv3”, “PHKA2sv4”, “PHKA2sv6.1”, “PHKA2sv6.2”, and “PHKA2sv7” refer to splice variant isoforms of human PHKA2 protein, wherein the splice variants have the amino acid sequence set forth in SEQ ID NO 2 (for PHKA2sv3), SEQ ID NO 4 (for PHKA2sv4), SEQ ID NO 6 (for amino terminal PHKA2sv6.1) and SEQ ID NO 8 (for carboxy terminal PHKA2sv6.2).SEQ ID NO 10 (for PHKA2sv7),

As used herein, “PHKA2sv3” refers to polynucleotides encoding PHKA2sv3 having an amino acid sequence set forth in SEQ ID NO 2. As used herein, “PHKA2sv4” refers to polynucleotides encoding PHKA2sv4 having an amino acid sequence set forth in SEQ ID NO 4. As used herein, “PHKA2sv6.1” refers to polynucleotides encoding PHKA2sv6.1 having an amino acid sequence set forth in SEQ ID NO 8. As used herein, “PHKA2sv6.2” refers to polynucleotides encoding PHKA2sv6.2 having an amino acid sequence set forth in SEQ ID NO 8. As used herein, “PHKA2sv7” refers to polynucleotides encoding PHKA2sv7 having an amino acid sequence set forth in SEQ ID NO 10.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurning forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

The present invention relates to the nucleic acid sequences encoding human PHKA2sv3, PHKA2sv4, PHKA2sv6.1, PHKA2sv6.2 and PHKA2sv7 that are alternatively spliced isoforms of PHKA2, and to the amino acid sequences encoding these proteins. SEQ ID NO 1, SEQ ID NO 3, SEQ ID NO 5, SEQ ID NO 7 and SEQ ID NO 9 are polynucleotide sequences representing the open reading frames that encode the PHKA2sv3, PHKA2sv4, PHKA2sv6.1, PHKA2sv6.2, and PHKA2sv7 proteins, respectively. SEQ ID NO 2 shows the polypeptide sequence of PHKA2sv3. SEQ ID NO 4 shows the polypeptide sequence of PHKA2sv4. SEQ ID NO 6 shows the polypeptide sequences of PHKA2sv6.1. SEQ ID NO 8 shows the polypeptide sequence of PHKA2sv6.2. SEQ ID NO 10 shows the polypeptide sequence of PHKA2sv7.

PHKA2sv3, PHKA2sv4, PHKA2sv6.1, PHKA2sv6.2 and PHKA2sv7 polynucleotide sequences encoding PHKA2sv3, PHKA2sv4, PHKA2sv6.1, PHKA2sv6.2, and PHKA2sv7 proteins, respectively, as exemplified and enabled herein include a number of specific, substantial and credible utilities. For example, PHKA2sv3, PHKA2sv4, PHKA2sv6.1, PHKA2sv6.2 and PHKA2sv7 encoding nucleic acids were identified in a mRNA sample obtained from a human source (see Example 1). Such nucleic acids can be used as hybridization probes to distinguish between cells that produce PHKA2sv3, PHKA2sv4, PHKA2sv6.1, PHKA2sv6.2 and PHKA2sv7 transcripts from human or non-human cells (including bacteria) that do not produce such transcripts. Similarly, antibodies specific for PHKA2sv3, PHKA2sv4, PHKA2sv6svl, PHKA2sv6.2 or PHKA2sv7 can be used to distinguish between cells that express PHKA2sv3, PHKA2sv4, PHKA2sv6.1, PHKA2sv6.2 or PHKA2sv7 from human or non-human cells (including bacteria) that do not express PHKA2sv3, PHKA2sv4, PHKA2sv6.1, PHKA2sv6.2 or PHKA2sv7.

PHKA2 is an important drug target for compounds that have therapeutic value in the management of glycogen and glucose levels. For example, curcumin has already been identified as a non-competitive and selective inhibitor of PHK enzyme activity (Reddy and Aggarwal, 1994 FEBS Letters 341:19-22), although it is not highly specific for PHKA2. Given the potential importance of PHKA2 activity to the therapeutic management of glycogen and glucose levels it is of value to identify PHKA2 isoforms and identify PHKA2-ligand compounds that are isoform-specific as well as compounds that are effective ligands for many PHKA2 isoforms. In particular, it may be important to identify compounds that are effective inhibitors of a specific PHKA2 isoform activity, yet do not bind to a plurality of other PHKA2 isoforms. Compounds that bind to multiple PHKA2 isoforms may require higher drug doses to saturate multiple PHKA2 isoform-binding sites, and thereby result in a greater likelihood of secondary non-therapeutic side effects. For the foregoing reasons, the PHKA2sv3, PHKA2sv4, PHKA2sv6.1, PHKAsv6.2 and PHKA2sv7 proteins represent useful compound binding targets and have utility in the identification of new PHKA2 ligands exhibiting a preferred specificity profile and having greater efficacy for their intended use.

In some embodiments, PHKA2sv3, PHKA2sv4, PHKA2sv6.1, PHKA2sv6.2 and PHKA2sv7 activity is modulated by a ligand compound to achieve one or more of the following: prevent or reduce the risk of occurrence or reoccurrence of X-linked glycogenesis and other metabolic diseases, including diabetes and obesity. Compounds that treat diabetes are particularly important because of the cause-and-effect relationship between diabetes and morbidity and mortality from its associated hypercholesterolemia, hypertriglyceridemia, atherosclerosis, and ulceration and gangrene of the extremities (For a review, Davis and Granner, In, Goodman & Gilman's The Pharmacological Basis of Therapeutics, 9th Ed., McGraw-Hill, New York, 1996, Ch. 61, pp. 1679-1714).

Compounds modulating PHKA2sv3, PHKA2sv4, PHKA2sv6.1, PHKA2sv6.2 or PHKA2sv7 include agonists, antagonists, and allosteric modulators. Generally, but not always, PHKA2sv3, PHKA2sv4, PHKA2sv6.1, PHKA2sv6.2 or PHKA2sv7-antagonists and allosteric modulators negatively affecting PHKA2sv3, PHKA2sv4, PHKA2sv6.1, PHKA2sv6.2 or PHKA2sv7 activity will be used to inhibit PHKA2 activity thereby decreasing glycogen mobilization and blood glucose levels. Inhibitors of PHKA2 achieve clinical efficacy by a number of effects, including inhibition of glycogen mobilization, that results in a decrease in blood glucose levels, which is especially important for reduction of blood glucose levels in patients with diabetes and obesity. Generally, but not always, PHKA2sv3, PHKA2sv4, PHKA2sv6.1, PHKA2sv6.2 or PHKA2sv7 agonists and allosteric modulators increasing PHKA2sv3, PHKA2sv4, PHKA2sv6.1, PHKA2sv6.2 or PHKA2sv7 activity will be used to increase glycogen mobilization and blood glucose levels. Increasing PHKA2 activity can also achieve clinical efficacy by reducing glycogen storage in patients with a glycogen storage disease.

PHKA2sv3, PHKA2sv4, PHKA2sv6.1, PHKA2sv6.2 or PHKA2sv7 activity can also be affected by modulating the cellular abundance of transcripts encoding PHKA2sv3, PHKA2sv4, PHKA2sv6.1, PHKA2sv6.2 or PHKA2sv7, respectively. Compounds modulating the abundance of transcripts encoding PHKA2sv3, PHKA2sv4, PHKA2sv6.1, PHKA2sv6.2 or PHKA2sv7 include a cloned polynucleotide encoding PHKA2sv3, PHKA2sv4, PHKA2sv6.1, PHKA2sv6.2 or PHKA2sv7, respectively, that can express PHKA2sv3, PHKA2sv4, PHKA2sv6.1, PHKA2sv6.2 or PHKA2sv7 in vivo, antisense nucleic acids targeted to PHKA2sv3, PHKA2sv4, PHKA2sv6.1, PHKA2sv6.2 or PHKA2sv7 transcripts, and enzymatic nucleic acids, such as ribozymes and RNAi, targeted to PHKA2sv3, PHKA2sv4, PHKA2sv6 or PHKA2sv7 transcripts.

In some embodiments, PHKA2sv3, PHKA2sv4, PHKA2sv6.1, PHKA2sv6.2 and/or PHKA2sv7 activity is modulated to achieve a therapeutic effect upon diseases. For example, diabetes may be treated by modulating PHKA2sv3, PHKA2sv4, PHKA2sv6.1, PHKA2sv6.2 or PHKA2sv7 activity to achieve, for instance, decreased levels of blood glucose. In other embodiments, X-linked glycogenosis is reduced by modulating PHKA2sv3, PHKA2sv4, PHKA2sv6.1, PHKA2sv6.2 or PHKA2sv7 activity to achieve, for example, increased levels of PHK activity.

PHKA2sv3, PHKA2sv4, PHKA2sv6.1, PHKA26.2 and PHKA2sv7 Nucleic Acid

PHKA2sv3 nucleic acids contain regions that encode for polypeptides comprising, consisting, or consisting essentially of SEQ ID NO 2. PHKA2sv4 nucleic acids contain regions that encode for polypeptides comprising, consisting, or consisting essentially of SEQ ID NO 4. PHKA2sv6.1 nucleic acids contain regions that encode for polypeptides comprising, consisting, or consisting essentially of SEQ ID NO 6. PHK42sv6.2 nucleic acids contain regions that encode for polypeptides comprising, consisting, or consisting essentially of SEQ ID NO 8. PHKA2sv7 nucleic acids contain regions that encode for polypeptides comprising, consisting, or consisting essentially of SEQ ID NO 10. The PHKA2sv3, PHKA2sv4, PHKA2sv6.1, PHKA2sv6.2 and PHKA2sv7 nucleic acids have a variety of uses, such as being used as a hybridization probe or PCR primer to identify the presence PHKA2sv3, PHKA2sv4, PHKA2sv6.1, PHKA2sv6.2 or PHKA2sv7 nucleic acid, respectively; being used as a hybridization probe or PCR primer to identify nucleic acid encoding for proteins related to PHKA2sv3, PHKA2sv4, PHKA2sv6.1, PHKA2sv6.2 or PHKA2sv7, respectively; and/or being used for recombinant expression of PHKA2sv3, PHKA2sv4, PHKA2sv6.1, PHKA2sv6.2 or PHKA2sv7 polypeptides, respectively. In particular, PHKA2sv3 polynucleotides do not have polynucleotide regions that comprises exons 27, 28 and 29 of the PHKA2 gene. PHKA2sv4 polynucleotides do not have the polynucleotide regions that comprises exon 16 of the PHKA2 gene. PHKA2sv6.1 and PHKA2sv6.2 polynucleotides have an additional polynucleotide region that comprises intron 16 of the PHKA2 gene. PHKA2sv7 polynucleotides do not have polynucleotide regions that comprises exon 7 of the PHKA2 gene.

Regions in PHKA2sv3, PHKA2sv4, PHKA2sv6, PHKA2sv6.2 or PHKA2sv7 nucleic acid that do not encode for PHKA2sv3, PHKA2sv4, PHKA2sv6.1, PHKA2sv6.2 or PHKA2sv7 or are not found in SEQ ID NO 1, SEQ ID NO 3, SEQ ID NO 5, SEQ ID NO 7 or SEQ ID NO 9 if present, are preferably chosen to achieve a particular purpose. Examples of additional regions that can be used to achieve a particular purpose include capture regions that can be used as part of an ELISA sandwich assay, reporter regions that can be probed to indicate the presence of the nucleic acid, expression vector regions, and regions encoding for other polypeptides.

The guidance provided in the present application can be used to obtain the nucleic acid sequence encoding PHKA2sv3, PHKA2sv4, PHKA2sv6.1, PHKA2sv6.2 or PHKA2sv7 related proteins from different sources. Obtaining nucleic acids encoding PHKA2sv3, PHKA2sv4, PHKA2sv6.1, PHKA2sv6.2 or PHKA2sv7 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{d} Edition, Cold Spring Harbor Laboratory Press, 1989. PCR cloning techniques are described, for example, in White, Methods in Molecular Cloning, volume 67, Humana Press, 1997.

PHKA2sv3, PHKA2sv4, PHKA2sv6.1, PHKA2sv6.2 or PHKA2sv7 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

PHKA2sv3, PHKA2sv4, PHKA2sv6.1, PHKA2sv6.2 and PHKA2sv7 Probes

Probes for PHKA2sv3, PHKA2sv4, PHKA2sv6.1, PHKA2sv6.2 or PHKA2sv7 contain a region that can specifically hybridize to PHKA2sv3, PHKA2sv4, PHKA2sv6.1, PHKA2sv6.2 or PHKA2sv7 target nucleic acids, respectively, under appropriate hybridization conditions and can distinguish PHKA2sv3, PHKA2sv4, PHKA2sv6.1, PHKA2sv6.2 or PHKA2sv7 nucleic acids from each other and from non-target nucleic acids, in particular PHKA2 polynucleotides containing exons 7, 16, 27, 28 and 29 and PHKA2 polynucleotides lacking intron 16. Probes for PHKA2sv3, PHK42sv4, PHKA2sv6.1, PHKA2sv6.2 or PHKA2sv7 can also contain nucleic acid regions that are not PHKA2sv3, PHKA2sv4, PHKA2sv6.1, PHKA2sv6.2 or PHKA2sv7 nucleic acids.

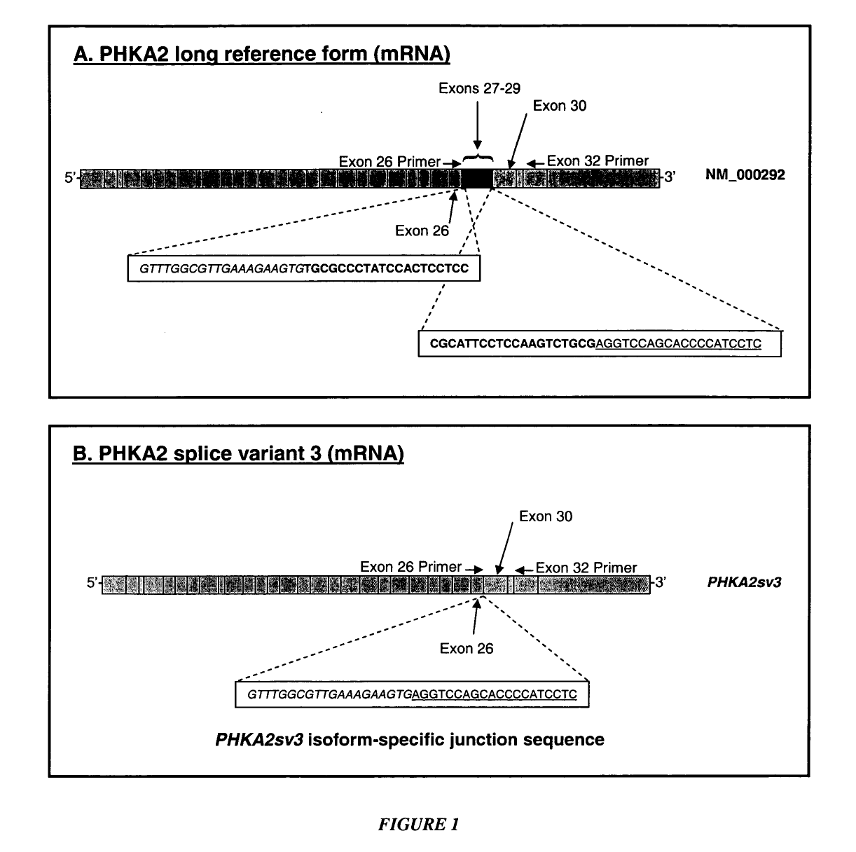

In embodiments where, for example, PHKA2sv3, PHKA2sv4, PHKA2sv6.1, PHKA2sv6.2 or PHKA2sv7 polynucleotide probes are used in hybridization assays to specifically detect the presence of PHKA2sv3, PHKA2sv4, PHKA2sv6.1, PHKA2sv6.2 or PHKA2sv7 polynucleotides in samples, the PHKA2sv3, PHKA2sv4, PHKA2sv6.1, PHKA2sv6.2 or PHKA2sv7 polynucleotides comprise at least 20 nucleotides of the PHKA2sv3, PHKA2sv4, PHKA2sv6.1, PHKA2sv6.2 or PHKA2sv7 sequence that correspond to the respective novel exon junction polynucleotide regions. In particular, for detection of PHKA2sv3, the probe comprises at least 20 nucleotides of the PHKA2sv3 sequence that corresponds to an exon junction polynucleotide created by the alternative splicing of exon 26 to exon 30 of the primary transcript of the PHKA2 gene (see FIG. 1B). For example, the polynucleotide sequence: 5′ GAAAGAA GTGAGGTCCAGCA 3′ [SEQ ID NO 11] represents one embodiment of such an inventive PHKA2sv3 polynucleotide wherein a first 10 nucleotides region is complementary and hybridizable to the 3′ end of exon 26 of the PHKA2 gene and a second 10 nucleotide region is complementary and hybridizable to the 5′ end of exon 30 of the PHKA2 gene (see FIG. 1B).

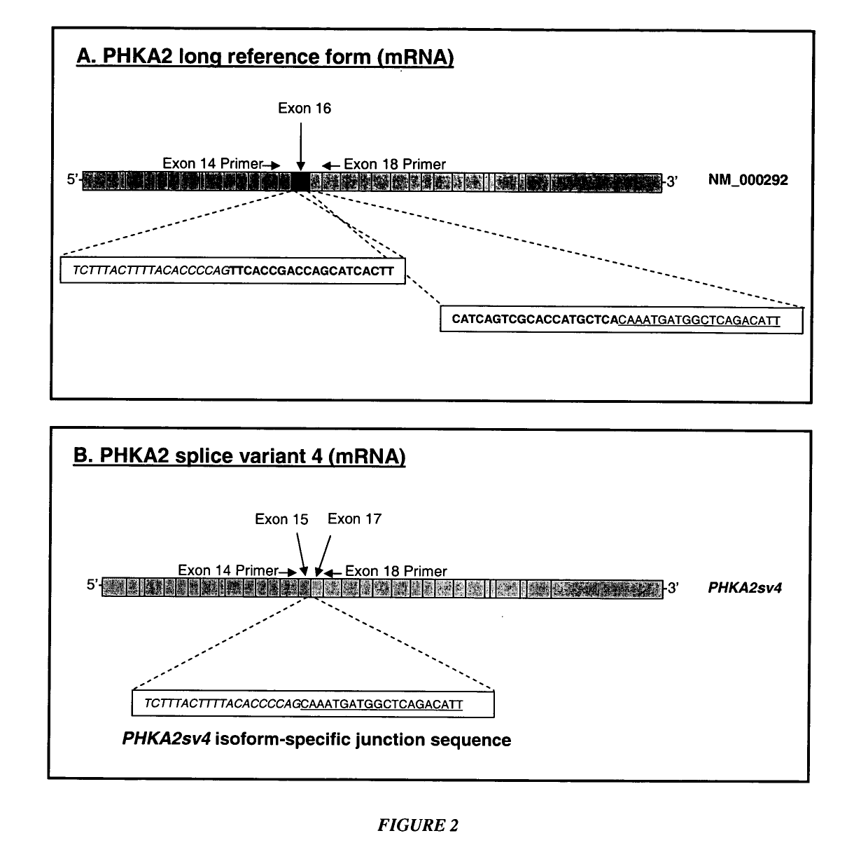

In another embodiment, for detection of PHKA2sv4, the probe comprises at least 20 nucleotides of the PHKA2sv4 sequence that corresponds to an exon junction polynucleotide created by the alternative splicing of exon 15 to exon 17 of the primary transcript of the PHKA2 gene (see FIG. 2B). For example, the polynucleotide sequence: 5′ TACACCCCAGCAAAT GATGG 3′ [SEQ ID NO 12] represents one embodiment of such an inventive PHKA2sv4 polynucleotide wherein a first 10 nucleotides region is complementary and hybridizable to the 3′ end of exon 15 of the PHKA2 gene and a second 10 nucleotide region is complementary and hybridizable to the 5′ end of exon 17 of the PHKA2 gene (see FIG. 2B).

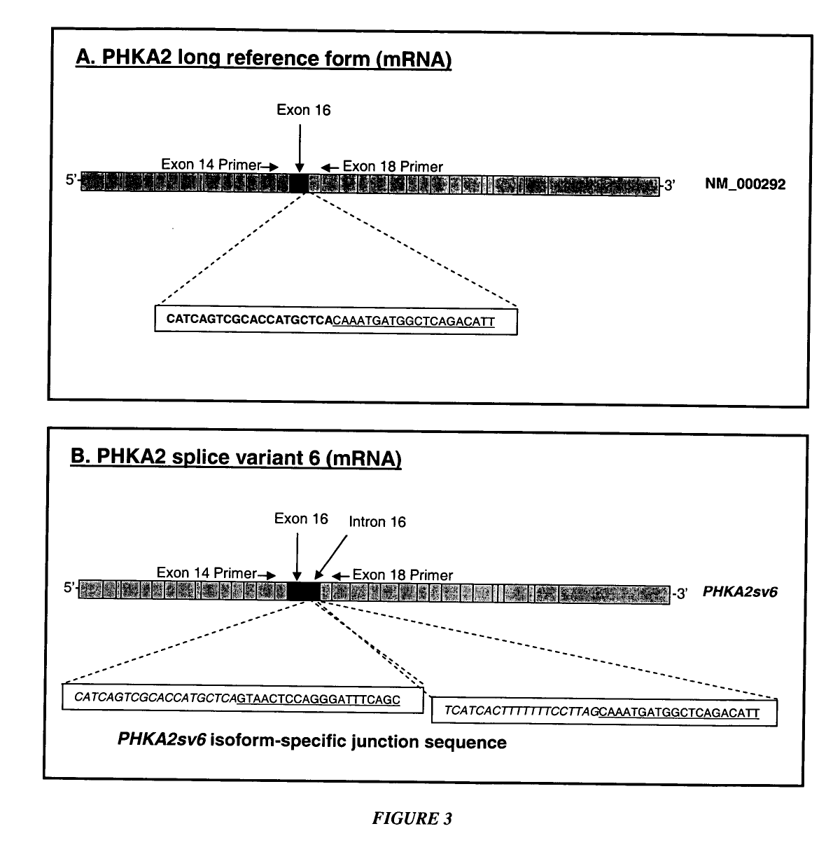

In another embodiment, for detection of PHKA2sv6.1 and PHKA2sv6.2, the probe comprises at least 20 nucleotides of the PHKA2sv6.1 sequence that corresponds to an exon junction polynucleotide created by the alternative splicing of exon 16 to intron 16 of the primary transcript of the PHKA2 gene (see FIG. 3B). For example, the polynucleotide sequence: 5′ ACCATGCTCAGTAACTCCAG 3′ [SEQ ID NO 13] represents one embodiment of such an inventive PHKA2sv6.1 polynucleotide wherein a first 10 nucleotides region is complementary and hybridizable to the 3′ end of exon 16 of the PHKA2 gene and a second 10 nucleotide region is complementary and hybridizable to the 5′ end of intron 16 of the PHKA2 gene (see FIG. 3B). In another example, the polynucleotide sequence: 5′ TTTTCCTTAGCAAATGATGG 3′ [SEQ ID NO 14] represents one embodiment of such an inventive PHKA2sv6.1 polynucleotide wherein a first 10 nucleotides region is complementary and hybridizable to the 3′ end of intron 16 of the PHKA2 gene and a second 10 nucleotide region is complementary and hybridizable to the 5′ end of exon 17 of the PHKA2 gene (see FIG. 3B).

In another embodiment, for detection of PHKA2sv6.2, the probe comprises at least 20 nucleotides of the PHKA2sv6.2 sequence shown in FIG. 15, beginning at nucleotide position 1 extending to position 101. For example, the polynucleotide sequence: 5′ ATGGTGT TGATGAAAATGTT 3′ [SEQ ID NO 15] represents one embodiment of such an inventive PHKA2sv6.2 polynucleotide wherein a 20 nucleotide region is complementary and hybridizable to a sequence that, in the prior art, is represented as being part of intron 16 of the PHKA2 gene.

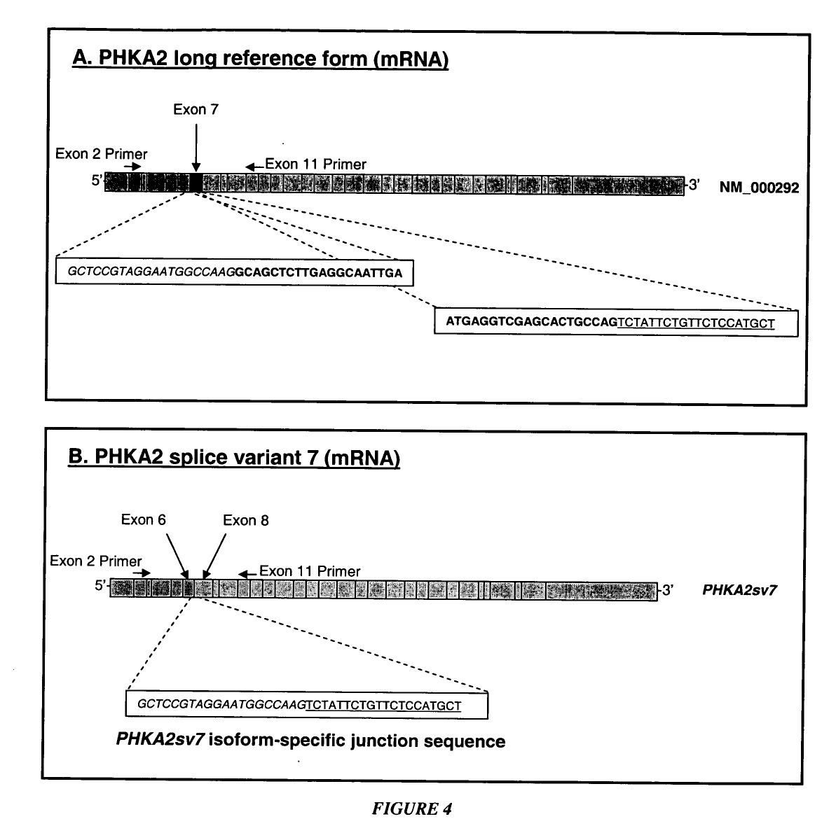

In another embodiment, for detection of PHKA2sv7, the probe comprises at least 20 nucleotides of the PHKA2sv7 sequence that corresponds to an exon junction polynucleotide created by the alternative splicing of exon 6 to exon 8 of the primary transcript of the PHKA2 gene (see FIG. 4A). For example, the polynucleotide sequence: 5′ AATGGCCAAGTCTAT TCTGT 3′ [SEQ ID NO 16] represents one embodiment of such an inventive PHKA2sv7 polynucleotide wherein a first 10 nucleotides region is complementary and hybridizable to the 3′ end of exon 6 of the PHKA2 gene and a second 10 nucleotide region is complementary and hybridizable to the 5′ end of exon 8 of the PHKA2 gene (see FIG. 4B).

In some embodiments, the first 20 nucleotides of PHKA2sv3 comprises a first continuous region of 5 to 15 nucleotides that is complementary and hybridizable to the 3′ end of exon 26 and a second continuous region of 5 to 15 nucleotides that is complementary and hybridizable to the 5′ end exon 30. In some embodiments, the first 20 nucleotides of PHKA2sv4 comprises a first continuous region of 5 to 15 nucleotides that is complementary and hybridizable to the 3′ end of exon 15 and a second continuous region of 5 to 15 nucleotides that is complementary and hybridizable to the 5′ end exon 17. In some embodiments, the first 20 nucleotides of PHKA2sv6.1 or PHKA2sv6.2 probes comprises a first continuous region of 5 to 15 nucleotides that is complementary and hybridizable to the 3′ end of exon 16 and a second continuous region of 5 to 15 nucleotides that is complementary and hybridizable to the 5′ end intron 16 of the PHKA2 gene or alternatively, the first 20 nucleotides of PHKA2sv6.1 or PHKA2sv6.2 comprises a first continuous region of 5 to 15 nucleotides that is complementary and hybridizable to the 3′ end of intron 16 and a second continuous region of 5 to 15 nucleotides that is complementary and hybridizable to the 5′ end exon 17 of the PHKA2 gene. In some embodiments, the first 20 nucleotides of PHKA2sv7 comprises a first continuous region of 5 to 15 nucleotides that is complementary and hybridizable to the 3′ end of exon 6 and a second continuous region of 5 to 15 nucleotides that is complementary and hybridizable to the 5′ end exon 8 of the PHKA2 gene.

In other embodiments, the PHKA2sv3, PHKA2sv4, PHKA2sv6.1, PHkA2sv6.2 or PHKA2sv7 polynucleotide comprise at least 40, 60, 80 or 100 nucleotides of the PHKA2sv3, PHKA2sv4, PHKA2sv6.1, PHKA2sv6.2 or PHKA42sv7 sequence, respectively, that correspond to a junction polynucleotide region created by the alternative splicing of exon 26 to exon 30 in the case of PHKA2sv3, the alternative splicing of exon 15 to exon 17 in the case of PHKA2sv4, the lack of splicing of exon 16 to exon 17 resulting in the retention of intron 16 in the case of PHKA42sv6.1 or PHKA2sv6.2, or in the case of PHKA2sv7, the alternative splicing of exon 6 to exon 8 of the primary transcript of the PHKA2 gene. In embodiments involving PHKA2sv3, the PHKA2sv3 polynucleotide is selected to comprise a first continuous region of at least 5 to 15 nucleotides that is complementary and hybridizable to the 3′ end of exon 26 and a second continuous region of at least 5 to 15 nucleotides that is complementary and hybridizable to the 5′ end of exon 30 of the PHKA2 gene. Similarly, in embodiments involving PHKA2sv4, the PHKA2sv4 polynucleotide is selected to comprise a first continuous region of at least 5 to 15 nucleotides that is complementary and hybridizable to the 3′ end of exon 15 and a second continuous region of at least 5 to 15 nucleotides that is complementary and hybridizable to the 5′ end of exon 17 of the PHKA2 gene. In embodiments involving PHKA2sv6.1, the PHKA2sv6.1 polynucleotide is selected to comprise a first continuous region of at least 5 to 15 nucleotides that is complementary and hybridizable to the 3′ end of exon 16 and a second continuous region of at least 5 to 15 nucleotides that is complementary and hybridizable to the 5′ end of intron 16 or the PHKA2sv6.1 polynucleotide is selected to comprise a first continuous region of at least 5 to 15 nucleotides that is complementary and hybridizable to the 3′ end of intron 16 and a second continuous region of at least 5 to 15 nucleotides that is complementary and hybridizable to the 5′ end of exon 17 of the PHKA2 gene. In other embodiments involving PHKA2sv6.2, the PHKA2sv6.2 polynucleotide is selected to comprise a continuous region of at least 15 nucleotides that is complementary and hybridizable to a sequence beginning at position 1 of SEQ ID NO: 7, or alternatively, the PHKA2sv6.1 polynucleotide is selected to comprise a first continuous region of at least 5 to 15 nucleotides that is complementary and hybridizable to the 3′ end of intron 16 and a second continuous region of at least 5 to 15 nucleotides that is complementary and hybridizable to the 5′ end of exon 17 of the PHKA2 gene. Similarly, in embodiments involving PHKA2sv7, the PHKA2sv7 polynucleotide is selected to comprise a first continuous region of at least 5 to 15 nucleotides that is complementary and hybridizable to the 3′ end of exon 6 and a second continuous region of at least 5 to 15 nucleotides that is complementary and hybridizable to the 5′ end of exon 8 of the PHKA2 gene. As will be apparent to a person of skill in the art, a large number of different polynucleotide sequences from the region of the exon 26 to exon 30 splice junction, the exon 15 to exon 17 splice junction, the exon 16 to intron 16 or intron 16 to exon 17 splice junction, or exon 6 to exon 8 splice junction may be selected which will, under appropriate hybridization conditions, have the capacity to detectably hybridize to PHKA2sv3, PHKA2sv4, PHKA2sv6.1, PHKA2sv6.2 or PHKA2sv7 polynucleotides, respectively, and yet will hybridize to a much less extent or not at all to PHKA2 isoform polynucleotides wherein exon 26 is not spliced to exon 30, wherein exon 15 is not spliced to exon 17, wherein exon 16 is not spliced to intron 16 or wherein intron 16 is not spliced to exon 17, or wherein exon 6 is not spliced to exon 8.

Preferably, non-complementary nucleic acid that is present has a particular purpose such as being a reporter sequence or being a capture sequence. However, additional nucleic acid need not have a particular purpose as long as the additional nucleic acid does not prevent the PHKA2sv3, PHKA2sv4, PHKA2sv6.1, PHKA2sv6.2 or PHKA2sv7 nucleic acid from distinguishing between target polynucleotides, e.g., PHKA2sv3, PHKA2sv4, PHKA2sv6.1, PHKA2sv6.2 or PHKA2sv7 polynucleotides and non-target polynucleotides, including, but not limited to PHKA2 polynucleotides not comprising the exon 26 to exon 30 splice junction, the exon 15 to exon 17, the exon 16 to intron 16 or intron 16 to exon 17, or exon 6 to exon 8 splice junctions found in PHKA2sv3, PHKA2sv4, PHKA2sv6.1, PHKA2sv6.2 or PHKA2sv7, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

PHKA2sv3, PHKA2sv4, PHKA2sv6.1, PHKA2sv6.2 or PHKA2sv7 polynucleotides, such as those comprising SEQ ID NO 1, SEQ ID NO 3, SEQ ID NO 5, SEQ ID NO 7 and SEQ ID NO 9, respectively, can be used to make PHKA2sv3, PHKA2sv4, PHKA2sv6.1, PHKA2sv6.2 or PHKA2sv7 polypeptides, respectively. In particular, make PHKA2sv3, PHKA2sv4, PHKA2sv6.1, PHKA2sv6.2 or PHKA2sv7 polypeptides can be expressed from recombinant nucleic acid in a suitable host or in vitro using a translation system. Recombinantly expressed PHKA2sv3, PHKA2sv4, PHKA2sv6.1, PHKA2sv6.2 or PHKA2sv7 polypeptides can be used, for example, in assays to screen for compounds that bind to make PHKA2sv3, PHKA2sv4, PHKA2sv6.1, PHKA2sv6.2 or PHKA2sv7, respectively. Alternatively, PHKA2sv3, PHKA2sv4, PHKA2sv6.1, PHKA2sv6.2 or PHKA2sv7 polypeptides can also be used to screen for compounds that bind to one or more PHKA2 isoforms but do not bind to PHKA2sv3, PHKA2sv4, PHKA2sv6.1, PHKA2sv6.2 or PHKA2sv7, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMClneo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

To enhance expression in a particular host it may be useful to modify the sequence provided in SEQ ID NO 1, SEQ ID NO 3, SEQ ID NO 5 or SEQ ID NO 6 to take into account codon usage of the host. Codon usage of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acid encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

PHKA2sv3, PHKA2sv4, PHKA2sv6.1, PHKA2sv6.2 and PHKA2sv7 Polypeptides

PHKA2sv3, PHKA2sv4, PHKA2sv6.1, PHKA2sv6.2 or PHKA2sv7 polypeptides contain an amino acid sequence comprising, consisting, or consisting essentially of SEQ ID NO 2, SEQ ID NO 4, SEQ ID NO 6, SEQ ID NO 8, or SEQ ID NO 10, respectively. PHKA2sv3, PHKA2sv4, PHKA2sv6.1, PHKA2sv6.2 or PHKA2sv7 polypeptides have a variety of uses, such as providing a marker for the presence of PHKA2sv3, PHKA2sv4, PHKA2sv6.1, PHKA2sv6.2 or PHKA2sv7, respectively; being used as an immunogen to produce antibodies binding to PHKA2sv3, PHKA2sv4, PHKA2sv6.1, PHKA2sv6.2 or PHKA2sv7, respectively; being used as a target to identify compounds binding selectively to PHKA2sv3, PHKA2sv4, PHKA2sv6.1, PHKA2sv6.2 or PHKA2sv7, respectively; or being used in an assay to identify compounds that bind to one or more iosforrns of PHKA2 but do not bind to or interact with PHKA2sv3, PHKA2sv4, PHKA2sv6.1, PHKA2sv6.2 or PHKA2sv7, respectively.

In chimeric polypeptides containing one or more regions from PHKA2sv3, PHKA2sv4, PHKA2sv6.1, PHKA2sv6.2 or PHKA2sv7 and one or more regions not from PHKA2sv3, PHKA2sv4, PHKA2sv6.1, PHKA2sv6.2 or PHKA2sv7, respectively, the region(s) not from PHKA2sv3, PHKA2sv4, PHKA2sv6.1, PHKA2sv6.2 or PHKA2sv7, respectively, can be used, for example, to achieve a particular purpose or to produce a polypeptide that can substitute for PHKA2sv3, PHKA2sv4, PHKA2sv6.1, PHKA2sv6.2 or PHKA2sv7 or fragments thereof. Particular purposes that can be achieved using chimeric PHKA2sv3, PHKA2sv4, PHKA2sv6.1, PHKA2sv6.2 or PHKA2sv7 polypeptides include providing a marker for PHKA2sv3, PHKA2sv4, PHKA2sv6.1, PHKA2sv6.2 or PHKA2sv7 activity, respectively, enhancing an immune response, and modulating glucose and/or glycogen levels.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional PHKA2sv3, PHKA2sv4, PHKA2sv6.1, PHKA2sv6.2 and PHKA2sv7

Functional PHKA2sv3, PHKA2sv4, PHKA2sv6.1, PHKA2sv6.2 and PHKA2sv7 are different protein isoforms of PHKA2. The identification of the amino acid and nucleic acid sequences of PHKA2sv3, PHKA2sv4, PHKA2sv6.1, PHKA2sv6.2 or PHKA2sv7 provide tools for obtaining functional proteins related to PHKA2sv3, PHKA2sv4, PHKA2sv6.1, PHKA2sv6.2 or PHKA2sv7, respectively, from other sources, for producing PHKA2sv3, PHKA2sv4, PHKA2sv6.1, PHKA2sv6.2 or PHKA2sv7 chimeric proteins, and for producing functional derivatives of SEQ ID NO 2, SEQ ID NO 4, SEQ ID NO 8, SEQ ID NO 9 or SEQ ID NO 7.

PHKA2sv3, PHKA2sv4, PHKA2sv6.1, PHKA2sv6.2 or PHKA2sv7 polypeptides can be readily identified and obtained based on their sequence similarity to PHKA2sv3 (SEQ ID NO 2), PHKA2sv4 (SEQ ID NO 4), PHKA2sv6.1 (SEQ ID NO 6), PHKA2sv6.2 (SEQ ID NO 8), or PHKA2sv7 (SEQ ID NO 10), respectively. In particular, PHKA2sv3 polypeptides lack the amino acids coded by exons 27, 28 and 29 of the PHKA2 gene. PHKA2sv4 polypeptides lack the amino acids coded by exon 16 of the PHKA2 gene. PHKA2sv6.1 polypeptide initiates translation at the canonical PHKA2 AUG start codon and contains additional amino acids, encoded by nucleotides located after the splice junction that result from the retention of intron 16 of the PHKA2 gene. This intron 16 sequence contains an in frame stop codon which results in a PHKA2sv6.1 polypeptide that is shorter than the PHKA2 reference polypeptide. PHKA2sv6.2 polypeptide initiates translation at an AUG start codon located in the retained intron 16 polynucleotides and results in a polypeptide that shares the same 662 carboxy-terminal amino acids of the reference PHKA2 protein and contains a unique 28 amino acid amino-terminal extension that is not similar to the PHKA2 reference polypeptide. Initiation at a downstream AUG of a bicistronic RNA is a fairly common event in eukaryotic cells and can be associated with disease (Meijer and Thomas, 2002 Biochem. J., 367: 1-11; Kozak, 2002 Mammalian Genome, 13:401-410). PHKA2sv7 polypeptides lack the amino acids coded by exon 7 of the PHKA2 gene.

Both the amino acid and nucleic acid sequences of PHKA2sv3, PHKA2sv4, PHKA2sv6.1, PHKA2sv6.2 or PHKA2sv7 can be used to help identify and obtain PHKA2sv3, PHKA2sv4, PHKA2sv6.1, PHKA2sv6.2 or PHKA2sv7 polypeptides, respectively. For example, SEQ ID NO 1 can be used to produce degenerative nucleic acid probes or primers for identifying and cloning nucleic acid polynucleotides encoding for a PHKA2sv3 polypeptide. In addition, polynucleotides comprising, consisting, or consisting essentially of SEQ ID NO 1 or fragments thereof, can be used under conditions of moderate stringency to identify and clone nucleic acid encoding PHKA2sv3 polypeptides from a variety of different organisms. The same methods can also be performed with polynucleotides comprising, consisting, or consisting essentially of SEQ ID NO 3 or fragments thereof to identify and clone nucleic acids encoding PHKA2sv4. Furthermore, the same methods can be performed with polynucleotides comprising, consisting, or consisting essentially of SEQ ID NO 5 or fragments thereof to identify and clone nucleic acids encoding PHKA2sv6.1. The same methods can also be performed with polynucleotides comprising, consisting, or consisting essentially of SEQ ID NO 7 or fragments thereof to identify and clone nucleic acids encoding PHKA2sv6.2. The same methods can also be performed with polynucleotides comprising, consisting, or consisting essentially of SEQ ID NO 9 or fragments thereof to identify and clone nucleic acids encoding PHKA2sv7.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with PHKA2sv3, PHKA2sv4, PHKA2sv6.1, PHKA2sv6.2 or PHKA2sv7 obtained from a particular source, derivatives can be produced. Such derivatives include polypeptides with amino acid substitutions, additions and deletions. Changes to PHKA2sv3, PHKA2sv4, PHKA2sv6.1, PHKA2sv6.2 or PHKA2sv7 to produce a derivative having essentially the same properties should be made in a manner not altering the tertiary structure of PHKA2sv3, PHKA2sv4, PHKA2sv6.1, PHKA2sv6.2 or PHKA2sv7, respectively.

Differences in naturally occurring amino acids are due to different R groups. An R group effects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

PHKA2sv3, PHKA2sv4, PHKA2sv6.1, PHKA2sv6.2 and PHKA2sv7 Antibodies

Antibodies recognizing PHKA2sv3, PHKA2sv4, PHKA2sv6.1, PHKA2sv6.2 or PHKA2sv7 can be produced using a polypeptide containing SEQ ID NO 2, SEQ ID NO 4, SEQ ID NO 6, SEQ ID NO 8, or SEQ ID NO 10, respectively, or a fragment thereof as an immunogen. Preferably, a PHKA2sv3 polypeptide used as an immunogen consists of a polypeptide of SEQ ID NO 2 or a SEQ ID NO 2 fragment having at least 10 contiguous amino acids in length corresponding to the polynucleotide region representing the junction resulting from the splicing of exon 26 to exon 30 of the PHKA2 gene. When a PHKA2sv4 polypeptide is used as an immunogen, preferably it consists of a polypeptide derived from SEQ ID NO 4 or a SEQ ID NO 4 fragment, having at least 10 contiguous amino acids in length corresponding to a polynucleotide region representing the junction resulting from the splicing of exon 15 to exon 17 of the PHKA2 gene. When a PHKA2sv6.1 polypeptide is used as an immunogen, preferably it consists of a polypeptide derived from SEQ ID NO 6 or a SEQ ID NO 6 fragment, having at least 10 contiguous amino acids in length corresponding to a polynucleotide region representing the junction from exon 16 to intron 16 of the PHKA2 gene. When a PHKA2sv6.2 polypeptide is used as an immunogen, preferably it consists of a polypeptide derived from SEQ ID NO 8 or a SEQ ID NO 8 fragment, having at least 10 contiguous amino acids in length corresponding to a polynucleotide region representing the junction from intron 16 to exon 17. When a PHKA2sv7 polypeptide is used as an immunogen, preferably it consists of a polypeptide derived from SEQ ID NO 10 or a SEQ ID NO 10 fragment, having at least 10 contiguous amino acids in length corresponding to a polynucleotide region representing the junction resulting from the splicing of exon 6 to exon 8 of the PHKA2 gene.

In some embodiments where, for example, PHKA2sv3 polypeptides are used to develop antibodies that bind specifically to PHKA2sv3 and not to other isoforms of PHKA2, the PHKA2sv3 polypeptides comprise at least 10 amino acids of the PHKA2sv3 polypeptide sequence corresponding to a junction polynucleotide region created by the alternative splicing of exon 26 to exon 30 of the primary transcript the PHKA2 gene (see FIG. 1B). For example, the amino acid sequence: amino terminus-GVERSEVQHP-carboxy terminus [SEQ ID NO 17], represents one embodiment of such an inventive PHKA2sv3 polypeptide wherein a first 5 amino acid region is encoded by nucleotide sequence at the 3′ end of exon 26 of the PHKA2 gene and a second 5 amino acid region is encoded by the nucleotide sequence directly after the novel splice junction. Preferably, at least 10 amino acids of the PHKA2sv3 polypeptide comprises a first continuous region of 2 to 8 amino acids that is coded by nucleotides at the 3′ end of exon 26 and a second continuous region of 2 to 8 amino acids that is coded by nucleotides at the 5′ end exon 30.

In other embodiments where, for example, PHKA2sv4 polypeptides are used to develop antibodies that bind specifically to PHKA2sv4 and not to other PHKA2 isoforms, the PHKA2sv4 polypeptides comprise at least 10 amino acids of the PHKA2sv4 polypeptide sequence corresponding to a junction polynucleotide region created by the alternative splicing of exon 15 to exon 17 of the primary transcript of the PHKA2 gene (see FIG. 2B). For example, the amino acid sequence: amino terminus-TFTPQQMMAQ-carboxy terminus [SEQ ID NO 18], represents one embodiment of such an inventive PHKA2sv4 polypeptide wherein a first 5 amino acid region is coded by a nucleotide sequence at the 3′ end of exon 15 of the PHKA2 gene and a second 5 amino acid region is coded by a nucleotide sequence directly after the novel splice junction. Preferably, at least 10 amino acids of the PHKA2sv4 polypeptide comprises a first continuous region of 2 to 8 amino acids that is coded by nucleotides at the 3′ end of exon 15 and a second continuous region of 2 to 8 amino acids that is coded by nucleotides at the 5′ end exon 17.

In other embodiments where, for example, PHKA2sv6.1 or PHKA2sv6.2 polypeptides are used to develop antibodies that bind specifically to PHKA2sv6.1 or PHKA2sv6.2 and not to other PHKA2 isoforms, the PHKA2sv6.1 or PHKA2sv6.2 polypeptides comprise at least 10 amino acids of the PHKA2sv6.1 or PHKA2sv6.2 polypeptide sequences corresponding to a junction polynucleotide region created by the retention of intron 16 of the primary transcript of the PHKA2 gene (see FIG. 3B). For example, in the case of PHKA2sv6.1 [SEQ ID NO 8], the amino acid sequence: amino terminus-SRTMLSNSRD-carboxy terminus [SEQ ID NO 19], represents one embodiment of such an inventive PHKA2sv6.1 polypeptide wherein a first 5 amino acid region is coded by a nucleotide sequence at the 3′ end of exon 16 of the PHKA2 gene and a second 5 amino acid region is coded by a nucleotide sequence at the 5′ end of intron 16. Preferably, at least 10 amino acids of the PHKA2sv6.1 polypeptide comprises a first continuous region of 2 to 8 amino acids that is coded by nucleotides at the 3′ end of exon 16 and a second continuous region of 2 to 8 amino acids that is coded by nucleotides at the 5′ end intron 16. Alternatively, in the case of PHKA2sv6.2 [SEQ ID NO 9], the amino acid sequence: amino terminus-FFSLANDGSG-carboxy terminus [SEQ ID NO 20], represents one embodiment of such an inventive PHKA2sv6.2 polypeptide wherein a first 5 amino acid region is coded by a nucleotide sequence at the 3′ end of intron 16 of the PHKA2 gene and a second 5 amino acid region is coded by a nucleotide sequence at the 5′ end of exon 17. Preferably, at least 10 amino acids of the PHKA2sv6.2 polypeptide comprises a first continuous region of 2 to 8 amino acids that is coded by nucleotides at the 3′ end of intron 16 and a second continuous region of 2 to 8 amino acids that is coded by nucleotides at the 5′ end exon 17.

In other embodiments where, for example, PHKA2sv7 polypeptides are used to develop antibodies that bind specifically to PHKA2sv7 and not to other PHKA2 isoforms, the PHKA2sv7 polypeptides comprise at least 10 amino acids of the PHKA2sv7 polypeptide sequence corresponding to a junction polynucleotide region created by the alternative splicing of exon 6 to exon 8 of the primary transcript of the PHKA2 gene (see FIG. 4B). For example, the amino acid sequence: amino terminus-VGMAKSILFS-carboxy terminus [SEQ ID NO 21], represents one embodiment of such an inventive PHKA2sv7 polypeptide wherein a first 5 amino acid region is coded by a nucleotide sequence at the 3′ end of exon 6 of the PHKA2 gene and a second 5 amino acid region is coded by a nucleotide sequence directly after the novel splice junction. Preferably, at least 10 amino acids of the PHKA2sv7 polypeptide comprises a first continuous region of 2 to 8 amino acids that is coded by nucleotides at the 3′ end of exon 6 and a second continuous region of 2 to 8 amino acids that is coded by nucleotides at the 5′ end exon 8.

In other embodiments, PHKA2sv3-specific antibodies are made using a PHKA2sv4 polypeptide that comprises at least 20, 30, 40 or 50 amino acids of the PHKA2sv4 sequence that corresponds to a junction polynucleotide region created by the alternative splicing of exon 26 to exon 30 of the primary transcript of the PHKA2 gene. In each case the PHKA2sv3 polypeptides are selected to comprise a first continuous region of at least 5 to 15 amino acids that is coded by nucleotides at the 3′ end of exon 26 and a second continuous region of 5 to 15 amino acids that is coded by nucleotides directly after the novel splice junction.

In other embodiments, PHKA2sv4-specific antibodies are made using a PHKA2sv4 polypeptide that comprises at least 20, 30, 40 or 50 amino acids of the PHKA2sv4 sequence that corresponds to a junction polynucleotide region created by the alternative splicing of exon 15 to exon 17 of the primary transcript of the PHKA2 gene. In each case the PHKA2sv4 polypeptides are selected to comprise a first continuous region of at least 5 to 15 amino acids that is coded by nucleotides at the 3′ end of exon 15 and a second continuous region of 5 to 15 amino acids that is coded by nucleotides directly after the novel splice junction.

In other embodiments, PHKA2sv6.1-specific antibodies are made using a PHKA2sv6.1 polypeptide that comprises at least 20, 30, 40 or 50 amino acids of the PHKA2sv6.1 sequence that corresponds to a junction polynucleotide region created by the retention of intron 16 of the primary transcript of the PHKA2 gene. In one case the PHKA2sv6.1 polypeptides are selected to comprise a first continuous region of at least 5 to 15 amino acids that is coded by nucleotides at the 3′ end of exon 16 and a second continuous region of 5 to 15 amino acids that is coded by nucleotides directly after the novel junction in intron 16 of the PHKA2 gene.

In other embodiments, PHKA2sv6.2-specific antibodies are made using a PHKA2sv6.2 polypeptide that comprises at least 20, 30, 40 or 50 amino acids of the PHKA2sv6.2 sequence that corresponds to a junction polynucleotide region created by the retention of intron 16 of the primary transcript of the PHKA2 gene. In one case the PHKA2sv6.2 polypeptides are selected to comprise a first continuous region of at least 5 to 28 amino acids that is coded by nucleotides beginning at position 1 of SEQ ID NO 7 and a second continuous region of 5 to 15 amino acids that is coded by nucleotides directly after the novel junction created by splicing of intron 16 to exon 17 of the PHKA2 gene.

In other embodiments, PHKA2sv7-specific antibodies are made using a PHKA2sv7 polypeptide that comprises at least 20, 30, 40 or 50 amino acids of the PHKA2sv7 sequence that corresponds to a junction polynucleotide region created by the alternative splicing of exon 6 to exon 8 of the primary transcript of the PHKA2 gene. In each case the PHKA2sv7 polypeptides are selected to comprise a first continuous region of at least 5 to 15 amino acids that is coded by nucleotides at the 3′ end of exon 6 and a second continuous region of 5 to 15 amino acids that is coded by nucleotides directly after the novel splice junction.

Antibodies to PHKA2sv3, PHKA2sv4, PHKA2sv6.1 or PHKA2sv6.2 or PHKA2sv7 have different uses such as being used to identify the presence of PHKA2sv3, PHKA2sv4, PHKA2sv6.1 or PHKA2sv6.2 or PHKA2sv7, respectively, and to isolate PHKA2sv3, PHKA2sv4, PHKA2sv6.1 or PHKA2sv6.2 or PHKA2sv7 polypeptides, respectively. Identifying the presence of PHKA2sv3 can be used, for example, to identify cells producing PHKA2sv3. Such identification provides an additional source of PHKA2sv3 and can be used to distinguish cells known to produce PHKA2sv3 from cells that do not produce PHKA2sv3. For example, antibodies to PHKA2sv3 can distinguish human cells expressing PHKA2sv3 from human cells not expressing PHKA2sv3 or non-human cells (including bacteria) that do not express PHKA2sv3. Such PHKA2sv3 antibodies can also be used to determine the effectiveness of PHKA2sv3 ligands, using techniques well known in the art, to detect and quantify changes in the protein levels of PHKA2sv3 in cellular extracts, and in situ immunostaining of cells and tissues. In addition, the same above-described utilities also exist for PHKA2sv4-specific antibodies, PHKA2sv6.1-specific antibodies, PHKA2sv6.2-specific antibodies and PHKA2sv7-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

PHKA2sv3, PHKA2sv4, PHKA2sv6.1 or PHKA2sv6.2 and PHKA2sv7 Binding Assays

PHKA2sv3, PHKA2sv4, PHKA2sv6.1, PHKA2sv6.2, PHKA2sv7 or fragments thereof can be used in binding studies to identify compounds binding to or interacting with PHKA2sv3, PHKA2sv4, PHKA2sv6.1, PHKA2sv6.2 or PHKA2sv7 or fragments thereof, respectively. In one embodiment, the PHKA2sv3 or a fragment thereof can be used in binding studies with PHKA2 isoform protein or a fragment thereof, to identify compounds that: bind to or interact with PHKA2sv3 and other PHKA2 isoforms; bind to or interact with one or more other PHKA2 isoforms and not with PHKA2sv3. A similar series of compound screens can, of course, also be performed using PHKA2sv4, PHKA2sv6.1, PHKA2sv6.2 or PHKA2sv7, rather than, or in addition to PHKA2sv3. Such binding studies can be performed using different formats including competitive and non-competitive formats. Further competition studies can be carried out using additional compounds determined to bind to PHKA2sv3, PHKA2sv4, PHKA2sv6.1, PHKA2sv6.2, PHKA2sv7 or other PHKA2 isoforms.

The particular PHKA2sv3, PHKA2sv4, PHKA2sv6.1, PHKA2sv6.2 or PHKA2sv7 sequence involved in ligand binding can be readily identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using PHKA2sv3 expressed from a recombinant nucleic acid. Alternatively, recombinantly expressed PHKA2sv3 consists of the SEQ ID NO 2 amino acid sequence. In addition, binding studies are performed using PHKA2sv4 expressed from a recombinant nucleic acid. Alternatively, recombinantly expressed PHKA2sv4 consists of the SEQ ID NO 4 amino acid sequence. In addition, binding studies are performed using PHKA2sv6.1 expressed from a recombinant nucleic acid. Alternatively, recombinantly expressed PHKA2sv6.1 consists of the SEQ ID NO 6 amino acid sequence. Alternatively, binding studies are performed using PHKA2sv6.2 expressed from a recombinant nucleic acid. Recombinantly expressed PHKA2sv6.2 consists of the SEQ ID NO 8 amino acid sequence. In addition, binding studies are performed using PHKA2sv7 expressed from a recombinant nucleic acid. Alternatively, recombinantly expressed PHKA2sv7 consists of the SEQ ID NO 10 amino acid sequence.

Binding assays can be performed using individual compounds or preparations containing different numbers of compounds. A preparation containing different numbers of compounds having the ability to bind to PHKA2sv3, PHKA2sv4, PHKA2sv6.1, PHKA2sv6.2 or PHKA2sv7 can be divided into smaller groups of compounds that can be tested to identify the compound(s) binding to PHKA2sv3, PHKA2sv4, PHKA2sv6.1, PHKA2sv6.2 or PHKA2sv7, respectively.

Binding assays can be performed using recombinantly produced PHKA2sv3, PHKA2sv4, PHKA2sv6.1, PHKA2sv6.2 or PHKA2sv7 present in different environments. Such environments include, for example, cell extracts and purified cell extracts containing a PHKA2sv3, PHKA2sv4, PHKA2sv6.1, PHKA2sv6.2 or PHKA2sv7 recombinant nucleic acid; and also include, for example, the use of a purified PHKA2sv3, PHKA2sv4, PHKA2sv6.1, PHKA2sv6.2 or PHKA2sv7 polypeptide produced by recombinant means which is introduced into different environments.

In one embodiment of the invention, a binding method is provided for screening for a compound able to bind selectively to PHKA2sv3. The method comprises the steps: providing a PHKA2sv3 polypeptide comprising SEQ ID NO 2; providing a PHKA2 isoform polypeptide that is not PHKA2sv3, contacting the PHKA2sv3 polypeptide and the PHKA2 isoform polypeptide that is not PHKA2sv3 with a test preparation comprising one or more test compounds; and then determining the binding of the test preparation to the PHKA2sv3 polypeptide and to the PHKA2 isoform polypeptide that is not PHKA2sv3 wherein a compound which binds to the PHKA2sv3 polypeptide but does not bind to PHKA2 isoform polypeptide that is not PHKA2sv3 contains one or more compounds that selectively binds to PHKA2sv3.

In another embodiment of the invention, a binding method is provided for screening for a compound able to bind selectively to PHKA2sv4. The method comprises the steps: providing a PHKA2sv4 polypeptide comprising SEQ ID NO 4; providing a PHKA2 isoform polypeptide that is not PHKA2sv4, contacting the PHKA2sv4 polypeptide and the PHKA2 isoform polypeptide that is not PHKA2sv4 with a test preparation comprising one or more test compounds; and then determining the binding of the test preparation to the PHKA2sv4 polypeptide and to the PHKA2 isoform polypeptide that is not PHKA2sv4 wherein a compound which binds to the PHKA2sv4 polypeptide but does not bind to PHKA2 isoform polypeptide that is not PHKA2sv4 contains one or more compounds that selectively binds to PHKA2sv4.

In another embodiment of the invention, a binding method is provided for screening for a compound able to bind selectively to PHKA2sv6.1. The method comprises the steps: providing a PHKA2sv6.1 polypeptide comprising SEQ ID NO 6; providing a PHKA2 isoform polypeptide that is not PHKA2sv6.1, contacting the PHKA2sv6.1 polypeptide and the PHKA2 isoform polypeptide that is not PHKA2sv6.1 with a test preparation comprising one or more test compounds; and then determining the binding of the test preparation to the PHKA2sv6.1 polypeptide and to the PHKA2 isoform polypeptide that is not PHKA2sv6.1 wherein a compound which binds to the PHKA2sv6.1 polypeptide but does not bind to PHKA2 isoform polypeptide that is not PHKA2sv6.1 contains one or more compounds that selectively binds to PHKA2sv6.1.

In another embodiment of the invention, a binding method is provided for screening for a compound able to bind selectively to PHKA2sv6.2. The method comprises the steps: providing a PHKA2sv6 polypeptide comprising SEQ ID NO 8; providing a PHKA2 isoform polypeptide that is not PHKA2sv6.2, contacting the PHKA2sv6.2 polypeptide and the PHKA2 isoform polypeptide that is not PHKA2sv6.2 with a test preparation comprising one or more test compounds; and then determining the binding of the test preparation to the PHKA2sv6.2 polypeptide and to the PHKA2 isoform polypeptide that is not PHKA2sv6.2 wherein a compound which binds to the PHKA2sv6.2 polypeptide but does not bind to PHKA2 isoform polypeptide that is not PHKA2sv6.2 contains one or more compounds that selectively binds to PHKA2sv6.2.

In another embodiment of the invention, a binding method is provided for screening for a compound able to bind selectively to PHKA2sv7. The method comprises the steps: providing a PHKA2sv7 polypeptide comprising SEQ ID NO 10; providing a PHKA2 isoform polypeptide that is not PHKA2sv7, contacting the PHKA2sv7 polypeptide and the PHKA2 isoform polypeptide that is not PHKA2sv7 with a test preparation comprising one or more test compounds; and then determining the binding of the test preparation to the PHKA2sv7 polypeptide and to the PHKA2 isoform polypeptide that is not PHKA2sv7 wherein a compound which binds to the PHKA2sv7 polypeptide but does not bind to PHKA2 isoform polypeptide that is not PHKA2sv7 contains one or more compounds that selectively binds to PHKA2sv7.

In another embodiment of the invention, a binding method is provided for screening for a compound able to bind selectively to a PHKA2 isoform polypeptide that is not PHKA2sv3. The method comprises the steps: providing a PHKA2sv3 polypeptide comprising SEQ ID NO 2; providing a PHKA2 isoform polypeptide that is not PHKA2sv3, contacting the PHKA2sv3 polypeptide and the PHKA2 isoform polypeptide that is not PHKA2sv3 with a test preparation comprising one or more test compounds; and then determining the binding of the test preparation to the PHKA2sv3 polypeptide and the PHKA2 isoform polypeptide that is not PHKA2sv3, wherein a test preparation that binds the PHKA2 isoform polypeptide that is not PHKA2sv3 but does not bind the PHKA2sv3 contains a compound that selectively binds the PHKA2 isoform polypeptide that is not PHKA2sv3. Alternatively, the above method can be used to identify compounds that bind selectively to a PHKA2 isoform polypeptide that is not PHKA2sv4 by performing the method with PHKA2sv4 protein comprising SEQ ID NO 4. Alternatively, the above method can be used to identify compounds that bind selectively to a PHKA2 isoform polypeptide that is not PHKA2sv6.1 by performing the method with PHKA2sv6.1 protein comprising SEQ ID NO 6. Alternatively, the above method can be used to identify compounds that bind selectively to a PHKA2 isoform polypeptide that is not PHKA2sv6.2 by performing the method with PHKA2sv6.2 protein comprising SEQ ID NO 8. Alternatively, the above method can be used to identify compounds that bind selectively to a PHKA2 isoform polypeptide that is not PHKA2sv7 by performing the method with PHKA2sv7 protein comprising SEQ ID NO 10.

The above-described selective binding assays can also be performed with a polypeptide fragment of PHKA2sv3, PHKA2sv4, PHKA2sv6.1, PHKA2sv6.2 or PHKA2sv7, wherein the polypeptide fragment comprises at least 10 consecutive amino acids that are coded by a nucleotide sequence that bridges the junction created by the splicing of the 3′ end of exon 26 to the 5′ end of exon 30 in the case of PHKA2sv3, by the splicing of the 3′ end of exon 15 to the 5′ end of exon 17, in the case of PHKA2sv4, by the splicing of the 3′ end of exon 16 to the 5′ end of intron 16, in the case of PHKA2sv6.1, or by the splicing of the 3′ end of intron 16 to the 5′ end of exon 17, in the case of PHKA2sv6.2, or by the splicing of the 3′ end of exon 6 to the 5′ end of exon 8, in the case of PHKA2sv7. Similarly, the selective binding assays may also be performed using a polypeptide fragment of a PHKA2 isoform polypeptide that is not PHKA2sv3, PHKA2sv4, PHKA2sv6.1, PHKA2sv6.2 or PHKA2sv7 wherein the polypeptide fragment comprises at least 10 consecutive amino acids that are coded by: a) a nucleotide sequence that is contained within exon 27, 28, or 29, of the PHKA2 gene; b) a nucleotide sequence that is contained within exon 16 of the PHKA2 gene; c) a nucleotide sequence that is contained within intron 16 of the PHKA2 gene; d) a nucleotide sequence that is contained within exon 7 of the PHKA2 gene; or e) a nucleotide sequence that bridges the junction created by the splicing of the 3′ end of exon 15 to the 5′ end of exon 16, the splicing of the 3′ end of exon 26 to the 5′ end of exon 27, the splicing of the 3′ end of exon 27 to the 5′ end of exon 28, the splicing of the 3′ end of exon 27 to the 5′ end of exon 28, the splicing of the 3′ end of exon 28 to the 5′ end of exon 29, the splicing of the 3′ end of exon 16 to the 5′ end of exon 17, or the splicing of the 3′ end of exon 6 to the 5′ end of exon 7 of the PHKA2 gene.

PHKA2 Functional Assays

The identification of PHKA2sv3, PHKA2sv4, PHKA2sv6.1, PHKA2sv6.2 and PHKA2sv7 as splice variants of PHKA2 provides a means for screening for compounds that bind to PHKA2sv3, PHKA2sv4, PHKA2sv6.1, PHKA2sv6.2 and/or PHKA2sv7 protein thereby altering the ability of the PHKA2sv3, PHKA2sv4, PHKA2sv6.1, PHKA2sv6.2 and/or PHKA2sv7 polypeptide to be phosphorylated by cAMP-dependent kinase (cAMPK) or by PHK itself. Assays involving a functional PHKA2sv3, PHKA2sv4, PHKA2sv6.1, PHKA2sv6.2 or PHKA2sv7 polypeptide can be employed for different purposes such as selecting for compounds active at PHKA2sv3, PHKA2sv4, PHKA2sv6.1, PHKA2sv6.2 and/or PHKA2sv7, evaluating the ability of a compound to effect PHK activity of each respective splice variant polypeptide, and mapping the activity of different PHKA2sv3, PHKA2sv4, PHKA2sv6.1, PHKA2sv6.2 and PHKA2sv7 regions. PHKA2sv3, PHKA2sv4, PHKA2sv6.1, PHKA2sv6.2 and PHKA2sv7 activity can be measured using different techniques such as: detecting a change in the intracellular conformation of PHKA2sv3, PHKA2sv4, PHKA2sv6.1, PHKA2sv6.2 or PHKA2sv7; detecting a change in the intracellular location of PHKA2sv3, PHKA2sv4, PHKA2sv6.1, PHKA2sv6.2 or PHKA2sv7; detecting the amount of PHKA2sv3, PHKA2sv4, PHKA2sv6.1, PHKA2sv6.2 or PHKA2sv7 phosphorylation by cAMPK; measuring the levels of PHKA2sv3, PHKA2sv4, PHKA2sv6.1, PHKA2sv6.2 or PHKA2sv7 phosphorylation by PHK; or measuring the level of PHK activity of different PHKA2sv3, PHKA2sv4, PHKA2sv6.1, PHKA2sv6.2 or PHKA2sv7.

Recombinantly expressed PHKA2sv3, PHKA2sv4, PHKA2sv6.1, PHKA2sv6.2 and PHKA2sv7 can be used to facilitate determining whether a compound is active at PHKA2sv3, PHKA2sv4, PHKA2sv6.1, PHKA2sv6.2 and PHKA2sv7. For example, PHKA2sv3, PHKA2sv4, PHKA2sv6.1, PHKA2sv6.2 and PHKA2sv7 can be expressed by an expression vector in a cell line and used in a co-culture growth assay, such as described in WO 99/59037, to identify compounds that bind to PHKA2sv3, PHKA2sv4, PHKA2sv6.1, PHKA2sv6.2 and PHKA2sv7.

Techniques for measuring substrate phosphorylation by cAMPK are well known in the art (Beyer et al., Biol. Chem., 381:457-461; Ramachandran et al., 1987 J. Biol. Chem., 262:3210-3218; Chan et al., 1982 J. Biol. Chem., 257:3655-3659). In addition, an assay to measure the autophosphorylation of PHK has also been described (Singh et al., 1982 J. Biol. Chem., 257:13379-13384). Furthermore, protocols for measuring PHK activity are also available in the prior art (Chan et al., 1982 J. Biol. Chem., 257:3655-3659). Large varieties of other assays have been used to investigate the properties of PHK and therefore would also be applicable to the measurement of PHKA2sv3, PHKA2sv4, PHKA2sv6.1, PHKA2sv6.2 and PHKA2sv7 function.

PHKA2sv3, PHKA2sv4, PHKA2sv6.1, PHKA2sv6.2 and PHKA2sv7 functional assays can be performed using cells expressing PHKA2sv3, PHKA2sv4, PHKA2sv6.1, PHKA2sv6.2 or PHKA2sv7 at a high level contacted with individual compounds or preparations containing different compounds. A preparation containing different compounds where one or more compounds affect PHKA2sv3, PHKA2sv4, PHKA2sv6.1, PHKA2sv6.2 and/or PHKA2sv7 in cells over producing PHKA2sv3, PHKA2sv4, PHKA2sv6.1, PHKA2sv6.2 and/or PHKA2sv7 as compared to control cells containing expression vector lacking PHKA2sv3, PHKA2sv4, PA2sv6.1, PHKA2sv6.2 and/or PHKA2sv7 coding sequence, can be divided into smaller groups of compounds to identify the compound(s) affecting PHKA2sv3, PHKA2sv4, PHKA2sv6.1, PHKA2sv6.2 and PHKA2sv7 activity.

PHKA2sv3, PHKA2sv4, PHKA2sv6.1, PHKA2sv6.2 and PHKA2sv7 functional assays can be performed using recombinantly produced PHKA2sv3, PHKA2sv4, PHKA2sv6 and PHKA2sv7 present in different environments. Such environments include, for example, cell extracts and purified cell extracts containing PHKA2sv3, PHKA2sv4, PHKA2sv6.1, PHKA2sv6.2 and PHKA2sv7 expressed from recombinant nucleic acid and an appropriate membrane for the polypeptide; and the use of a purified PHKA2sv3, PHKA2sv4, PHKA2sv6.1, PHKA2sv6.2 and PHKA2sv7 produced by recombinant means that is introduced into a different environment suitable for measuring PHK activity.

Modulating PHKA2sv3, PHKA2sv4, PHKA2sv6.1 PHKA2sv6.2 and PHKA2sv7 Expression

PHKA2sv3, PHKA2sv4, PHKA2sv6.1, PHKA2sv6.2 or PHKA2sv7 expression can be modulated as a means for increasing or decreasing PHKA2sv3, PHKA2sv4, PHKA2sv6.1, PHKA2sv6.2 or PHKA2sv7 activity, respectively. Such modulation includes inhibiting the activity of nucleic acids encoding the PHKA2 isoform target to reduce PHKA2 isoform protein or polypeptide expressions, or supplying PHKA2 nucleic acids to increase the level of expression of the PHKA2 target polypeptide thereby increasing PHKA2 activity.

Inhibition of PHKA2sv3, PHKA2sv4, PHKA2sv6.1, PHKA2sv6.2 and PHKA2sv7 Activity

PHKA2sv3, PHKA2sv4, PHKA2sv6.1, PHKA2sv6.2 or PHKA2sv7 nucleic acid activity can be inhibited using nucleic acids recognizing PHKA2sv3, PHKA2sv4, PHKA2sv6.1, PHKA2sv6.2 or PHKA2sv7 nucleic acid and affecting the ability of such nucleic acid to be transcribed or translated. Inhibition of PHKA2sv3, PHKA2sv4, PHKA2sv6.1, PHKA2sv6.2 or PHKA2sv7 nucleic acid activity can be used, for example, in target validation studies.

A preferred target for inhibiting PHKA2sv3, PHKA2sv4, PHKA2sv6.1, PHKA2sv6.2 or PHKA2sv7 is mRNA translation. The ability of PHKA2sv3, PHKA2sv4, PHKA2sv6.1, PHKA2sv6.2 or PHKA2sv7 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acid can recognize and cleave another nucleic acid molecule. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can effect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999 Cell 99:123-32; Fire, et al., 1998 Nature 391:806-11), plants (Hamilton and Baulcombe, 1999 Science 286:950-52), Drosophila (Hammond, et al., 2001 Science 293:1146-50; Misquitta and Patterson, 1999 Proc. Nat. Acad. Sci. 96:1451-56; Kennerdell and Carthew, 1998 Cell 95:1017-26), and mammalian cells (Bernstein, et al., 2001 Nature 409:363-6; Elbashir, et al., 2001 Nature 411:494-8).

Increasing PHKA2sv3, PHKA2sv4, PHKA2sv6.1, PHKA2sv6.2 and PHKA2sv7 Expression

Nucleic acid coding for PHKA2sv3, PHKA2sv4, PHKA2sv6.1, PHKA2sv6.2 or PHKA2sv7 can be used, for example, to cause an increase PHK activity or to create a test system (e.g., a transgenic animal) for screening for compounds affecting PHKA2sv3, PHKA2sv4, PHKA2sv6.1, PHKA2sv6.2 or PHKA2sv7 expression, respectively. Nucleic acids can be introduced and expressed in cells present in different environments.

Guidelines for pharmaceutical administration in general are provided in, for example, Remington's Pharnaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of PHKA2sv3, PHKA2sv4 PHKA2sv6.1, PHKA2sv6.2 and PHKA2sv7 Using Microarrays

To identify variants of the “normal” splicing of the exon regions encoding PHKA2, an exon junction microarray, comprising probes complementary to each splice junction resulting from splicing of the 33 exon coding sequences in PHKA2 heteronuclear RNA (mRNA), was hybridized to a mixture of labeled nucleic acid samples prepared from 44 different human tissue or cell line samples. Exon junction microarrays are described in PCT patent applications WO 02/18646 and WO 02/16650. Materials and methods for preparing hybridization samples from purified RNA, hybridizing the microarrays, detecting hybridization signals, and data analysis are described in van't Veer, et al. (2002 Nature 415:530-536) and Hughes, et al. (2001 Nature Biotechnol. 19:342-7). Inspection of the microarray hybridization data (not shown) suggested that the structure of at least four of the exon junctions of PHKA2 mRNA were altered in some of the tissues examined, suggesting the presence of at least four PHKA2 splice variant mRNA populations within the “normal” PHKA2 mRNA population. RT-PCR was then performed using oligonucleotide primers complementary to exons 26 and 32, primers complimentary to exons 14 and 18, and primers complimentary to exons 2 and 11 to confirm the exon junction array results and to allow the sequence structure of the splice variants to be determined.

### Example 2

### Confirmation of PHKA2sv3, PHKA2sv4, PHKA2sv6.1, PHKA2sv6.2 and PHKA2sv7 Using RT-PCR

The structure of PHKA2 mRNA in the regions spanning exons 26 to 32, exons 14 to 18 and exons 2 to 11 was determined for a panel of human tissue and cell line samples using a RT-PCR based assay. PolyA enriched mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers of 28 nucleotides were selected that were complementary to sequences in exons 26 and 32, exons 14 and 18, and exons 2 and 11 of the reference exon coding sequences in PHKA2 (NM_{—}000292). Based upon the nucleotide sequence of PHKA2 mRNA, the PHKA2 exon 28 and exon 32 primer set (hereafter PHKA2₂₈₋₃₂ primer set) was expected to amplify a 521 basepair amplicon representing the “reference” PHKA2 mRNA region corresponding to exons 26 to 32 of the mRNA region for PHKA2sv3. In addition, the PHKA2 exon 14 and exon 18 primer set (hereafter PHKA2₁₄₋₁₈ primer set) was expected to amplify a 447 basepair amplicon representing the “reference” PHKA2 mRNA region corresponding to exons 14 to 18 of the mRNA region for PHKA2sv4. The PHKA2₁₄₋₁₈ primer set was expected to amplify a 447 basepair amplicon representing the “reference” PHKA2 mRNA region corresponding to exons 14 to 18 of the mRNA region for endogenous PHKA2sv6.1 and PHKA2sv6.2. The PHKA2 exon 2 and exon 11 primer set (hereafter PHKA2₂₋₁₁ primer set) was expected to amplify a 904 basepair amplicon representing the “reference” PHKA2 mRNA region corresponding to exons 2 to 11 of the mRNA region for PHKA2sv7. The PHKA2 exon 26 primer has the sequence: 5′ GAAAGTTTGATGAACCTCA GCCCTTTCG 3′ [SEQ ID NO 22]; and the PHKA2 exon 32 primer has the sequence: 5′ AAACTTGATCTCATGCGGGGTCATCTCT 3′ [SEQ ID NO 23]. The PHKA2 exon 14 primer has the sequence: 5′ AGAGTATCGCGGACATTCATCCAATTCA 3′ [SEQ ID NO 24]; and the PHKA2 exon 18 primer has the sequence: 5′ ACGATGTGGTGAGAAATTCCGA AAGGT 3′ [SEQ ID NO 25]. The PHKA2 exon 2 primer has the sequence: 5′ ATAACATCT ACAGTATCCTGGCCGTGTG 3′ [SEQ ID NO 26]; and the PHKA2 exon 11 primer has the sequence: 5′ TGAACAGCATCACCACTGAAGACTCCAT 3′ [SEQ ID NO 27].

Twenty-five ng of polyA enriched mRNA from each tissue or cell line sample was subjected to a one-step reverse transcription-PCR amplification protocol using the Qiagen, Inc. (Valencia, Calif.), One-Step RT-PCR kit, using the following conditions:

Cycling conditions were as follows:
50° C. for 30 minutes;
95° C. for 15 minutes;
35 cycles of:
   94° C. for 1 minute;
   60° C. for 1 minute;
   72° C. for 1 minute; then
72° C. for 10 minutes.

RT-PCR amplification products (amplicons) were size fractionated on a 2% agarose gel (data not shown). Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

Three different RT-PCR amplicons were obtained from human mRNA samples using the PHKA2₂₆₋₃₂ primer set (data not shown). Every sample assayed exhibited the expected amplicon size of 521 basepairs for normally spliced PHKA2 reference mRNA. However, in addition to the expected PHKA2 amplicon of 521 basepairs, many of the samples also exhibited a second amplicon of 345 basepairs. Interestingly, many mRNA samples appear to exhibit three different PHKA2 mRNA forms; the longer reference form, an intermediate sized form, and a shorter form amplicon of about 325 basepairs. The intermediate sized amplicon was observed in all mRNA samples where the short form was detected.

Four different RT-PCR amplicons were obtained from human mRNA samples using the PHKA2₁₄₋₁₈ primer set (data not shown). All tissues exhibited the expected amplicon size of 447 basepairs for the normally spliced reference PHKA2 mRNA. However, many samples also exhibited an amplicon of 300 basepairs. In addition, many samples also exhibited an amplicon of 693 basepairs.

Three different RT-PCR amplicons were obtained from human mRNA samples using the PHKA2₂₋₁₁ primer set (data not shown). All tissues exhibited the expected amplicon size of 904 basepairs for the normally spliced reference PHKA2 mRNA. However, many samples also exhibited an amplicon of 805 basepairs.

Sequence analysis of the 345 basepair amplicon of PHKA2 revealed that this amplicon form is due to splicing of exon 26 of the PHKA2 mRNA to exon 30. That is, the short form PHKA2 amplicon is due to the complete absence of exons 27, 28 and 29 nucleotide sequences. Thus, the RT-PCR results confirmed the microarray data reported in Example 1, which suggested that PHKA2 mRNA in some tissue mRNA samples is composed of a mixed population of molecules wherein in at least one of the PHKA2 mRNA populations, the splicing of exons 27, 28 and 29 is altered. This splice variant form was designated PHKA2sv3.

Sequence analysis of the 300 basepair amplicon of PHKA2 revealed that this amplicon form is due to splicing of exon 15 of the PHKA2 mRNA to exon 17. That is, the short form PHKA2 amplicon is due to the complete absence of the exon 16 polynucleotide sequence. Thus, the RT-PCR results confirmed the microarray data reported in Example 1, which suggested that PHKA2 mRNA in some tissue mRNA samples is composed of a mixed population of molecules wherein in at least one of the PHKA2 mRNA populations, the splicing of exon 16 is altered. This splice variant form was designated PHKA2sv4.

Sequence analysis of the 693 basepair amplicon of PHKA2 revealed that this amplicon form is due to the retention of intron 16 of the PHKA2 mRNA. That is, the longer form PHKA2 amplicon is due to the insertion of intron 16 polynucleotide sequence. Thus, the RT-PCR results confirmed the microarray data reported in Example 1, which suggested that PHKA2 mRNA in some tissue mRNA samples is composed of a mixed population of molecules wherein in at least one of the PHKA2 mRNA populations, the splicing of exon 16 is altered. This splice variant form was designated PHKA2sv6.

Sequence analysis of the 805 basepair amplicon of PHKA2 revealed that this amplicon form is due to the splicing of exon 6 of the PHKA2 mRNA to exon 8. That is, the short form PHKA2 amplicon is due to the complete absence of the exon 7 polynucleotide sequence. Thus, the RT-PCR results confirmed the microarray data reported in Example 1, which suggested that PHKA2 mRNA in some tissue mRNA samples is composed of a mixed population of molecules wherein in at least one of the PHKA2 mRNA populations, the splicing of exon 7 is altered. This splice variant form was designated PHKA2sv7.

Table 1 presents a summary of the presence or absence of splice variant PCR amplicons corresponding to PHKA2 splice variants sv3, sv4, sv6 and sv7 across 44 cell samples. The presence of an “X” in a column indicates that the corresponding splice variant PCR amplicon was visually detectable after staining of the size fractionated DNA amplification prducts.

TABLE 1

| Sample | PHKA2sv3 | PHKA2sv4 | PHKA2sv6 | PHKA2sv7 |
|---|---|---|---|---|
| Heart | | | X | |
| Kidney | | | X | X |
| Liver | | | X | X |
| Brain | X | X | X | X |
| Placenta | X | X | X | X |
| Lung | X | X | X | X |
| Fetal Brian | X | X | X | X |
| Leukemia Promyelocytic (HL-60) | X | | X | X |
| Adrenal Gland | X | X | X | X |
| Fetal Liver | X | | X | X |
| Salivary Gland | X | X | X | X |
| Pancreas | X | X | X | X |
| Skeletal Muscle | X | X | X | |
| Brain Cerebellum | X | X | X | X |
| Stomach | X | | X | X |
| Trachea | X | | X | X |
| Thyroid | X | X | X | X |
| Bone Marrow | X | X | X | X |
| Brain Amygdala | X | X | X | X |
| Brain Caudate Nucleus | X | X | X | X |
| Brain Corpus Callosum | X | X | X | X |
| Ileocecum | X | | X | X |
| Lymphoma Burkitt's (Raji) | X | | X | X |
| Spinal Cord | X | X | X | X |
| Lymph Node | X | X | X | X |
| Fetal Kidney | X | X | X | X |
| Uterus | X | X | X | X |
| Spleen | X | X | X | X |
| Brain Thalamus | X | X | X | X |
| Fetal Lung | X | X | X | X |
| Testis | X | X | X | X |
| Melanoma (G361) | X | X | X | X |
| Lung Carcinoma (A549) | X | X | X | X |
| Adrenal Medula, normal | | X | X | X |
| Brain, Cerebral Cortex, normal; | X | X | X | X |
| Descending Colon, normal | X | X | X | X |
| Prostate | X | | X | X |
| Duodenum, normal | X | | X | X |
| Epididymus, normal | X | | X | X |
| Brain, Hippocamus, normal | X | X | X | X |
| Ileum, normal | X | X | X | X |
| Interventricular Septum, normal | X | X | X | X |
| Jejunum, normal | X | X | X | X |
| Rectum, normal | X | X | X | X |

### Example 3

### Cloning of PHKA2sv3, PHKA2sv4 PHKA2sv6.1, PHKA2sv6.2 and PHKA2sv7

Microarray and RT-PCR data indicate that in addition to the normal PHKA2 reference mRNA sequence, NM_{—}000292, encoding PHKA2 protein, (NP_{—}000283), four novel splice variant forms of PHKA2 mRNA also exists in many tissues. Indeed, inspection of the amplicon band intensities in the agarose gels used to obtain the results displayed in Table 1, suggested that the 345 basepair PHKA2 short form of the PHKA2 mRNA is present in an amount that is about equal to or slightly less than the “reference” exon 27, 28, and 29 containing PHKA2 mRNA. Furthermore, inspection of the amplicon band intensities in FIG. 2, suggests that the 300 basepair PHKA2 short form of the PHKA2 mRNA is present in an amount about one fifth of the “reference” exon 16 containing PHKA2 mRNA. Inspection of the amplicon band intensities in the agarose gels used to obtain the results displayed in Table 1, suggests that the 693 basepair PHKA2 long form of the PHKA2 mRNA is present in an amount about one fifth of the “reference” intron 16 lacking PHKA2 mRNA. Furthermore, inspection of the amplicon band intensities in the agarose gels used to obtain the results displayed in Table 1, suggests that the 805 basepair PHKA2 short form of the PHKA2 mRNA is present in an amount about one fifth of the “reference” exon 7 containing PHKA2 mRNA.

A full length PHKA2 clone having a nucleotide sequence comprising the splice variants PHKA2sv3, PHKA2sv4, PHKA2sv6.1, PHKA2sv6.2 and PHKA2sv7, as identified in Example 2, are isolated using a 5′ “forward”, PHKA2 primer and a 3′ “reverse” PHKA2 primer, to amplify and clone the entire PHKA2sv3, PHKA2sv4, PHKA2sv6.1, PHKA2sv6.2 and PHKA2sv7mRNA coding sequences, respectively. The same 5′ “forward” primer is designed for isolation of full length clones corresponding to the PHKA2sv3, PHKA2sv4, PHKA2sv6.1 and PHKA2sv7 splice variants and has the nucleotide sequence of: 5′ ATGCGGAGCAGGAGC AATTCCGGGGTC 3′ [SEQ ID NO 28]. The 3′ “reverse” PHKA2sv3 primer is designed to have the nucleotide sequence of: 5° CCCCAGCCGATGTGATGTCCTCCCGAGT 3′ [SEQ ID NO 29]. The 3′ “reverse” primer for PHKA2sv4 is designed to have the nucleotide sequence of: 5′ GTTTTCTAATTGTGGAGAGCACAGCAGA 3′ [SEQ ID NO 30]. The 3′ “reverse” PHKA2sv6.1 primer is designed to have the nucleotide sequence of: 5′ AACATTTGTAAGAG CCCAAACCACCCCT 3′ [SEQ ID NO 31]. The 5′ “forward” PHKA2sv6.2 primer is designed to have the nucleotide sequence of: 5′ ATGGTGTTGATGAAAATGTTTCAGTGCA 3′ [SEQ ID NO 32] and the 3′ “reverse” PHKA2sv6.2 primer is designed to have the nucleotide sequence of: 5′ TTGCATCTGGCAGCCCGAATTGGGCAAC 3′ [SEQ ID NO 33]. The 3′ “reverse” primer for PHKA2sv7 is designed to have the nucleotide sequence of: 5′ TTGCATCTGGCAGC CCGAATTGGGCAAC 3′ [SEQ ID NO 34].

RT-PCR

The PHKA2sv3, PHKA2sv4, PHKA2sv6.1, PHKA2sv6.2, and PHKA2sv7 cDNA sequences are cloned using a combination of reverse transcription (RT) and polymerase chain reaction (PCR). More specifically, about 25 ng of human brain polyA mRNA (Ambion, Austin, Tex.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (PaloAlto, Calif.) Advantage 2 PCR kit. PCR is done in a Gene Amp PCR System 9700 (Applied Biosystems, Foster City, Calif.) using the PHKA2 “forward” and “reverse” primers. After an initial 94° C. denaturation of 1 minute, 35 cycles of amplification are performed using a 30 second denaturation at 94° C. followed by a 1 minute annealing at 65° C. and a 90 second synthesis at 68° C. The 35 cycles of PCR are followed by a 7 minute extension at 68° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determined if the PCR has yielded products of the expected size, in the case of the predicted PHKA2sv3, PHKA2sv4, PHKA2sv6.1, PHKA2sv6.2 and PHKA2sv7 mRNAs, products of about 3.0, 1.6, 1.8, 2.0, and 3.6 kilobases, respectively. The remainder of the 50 μl PCR reactions from human brain is purified using the QIAquik Gel extraction Kit (Qiagen, Valencia, Calif.) following the QIAquik PCR Purification Protocol provided with the kit. An about 50 μl of product obtained from the purification protocol is concentrated to about 6 μl by drying in a Speed Vac Plus (SC110A, from Savant, Holbrook, N.Y.) attached to a Universal Vacuum Sytem 400 (also from Savant) for about 30 minutes on medium heat.

Cloning of RT-PCR Products

About 4 μl of the 6 μl of purified PHKA2sv3, PHKA2sv4, PHKA2sv6.1, PHKA3sv6.2 and PHKA2sv7 RT-PCR products from human brain are used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μl/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative PHKA2sv3, PHKA2sv4, PHKA42sv6.1, PHKA42sv6.2 and PHKA2sv7 clones, respectively are identified and prepared for a PCR reaction to confirm the presence of the expected PHKA2sv3 exon 26 to exon 30, PHKA2sv4 exon 15 to exon 17, PHKA2sv6.1 exon 16 to intron 16, PHKA2sv6.2 intron 16 to exon 17 and PHKA2sv7 exon 6 to exon 8 splice variant structures. A 25 μl PCR reaction is performed as described above (RT-PCR section) to detect the presence of PHKA2sv3, except that the reaction includes miniprep DNA from the TOPO TA/PHKA2sv3 ligation as a template, and uses the PHKA2₂₆₋₃₀ primer set. An additional 25 μl PCR reaction is performed as described above (RT-PCR section) to detect the presence of PHKA2sv4, except that the reaction includes miniprep DNA from the TOPO TA/PHKA2sv4 ligation as a template, and uses the PHKA2₁₄₋₁₈ primer set. An additional 25 μl PCR reaction is performed as described above (RT-PCR section) to detect the presence of PHKA2sv6.1, except that the reaction includes miniprep DNA from the TOPO TA/PHKA2sv6.1 ligation as a template, and uses primers SEQ ID NO: 24 and SEQ ID NO 31 as a primer set. An additional 25 μl PCR reaction is performed as described above (RT-PCR section) to detect the presence of PHKA2sv6.2, except that the reaction includes miniprep DNA from the TOPO TA/PHKA2sv6.2 ligation as a template, and uses primers SEQ ID NO 32 and SEQ ID NO 25 as a primer set. An additional 25 μl PCR reaction is performed as described above (RT-PCR section) to detect the presence of PHKA2sv7, except that the reaction includes miniprep DNA from the TOPO TA/PHKA2sv7 ligation as a template, and uses the PHKA2₂₋₁₁ primer set. About 10 μl of each 25 μl PCR reaction is run on a 1% Agarose gel and the DNA bands generated by the PCR reaction are visualized and photographed on a UV light box to determine which minipreps samples have PCR product of the size predicted for the predicted corresponding PHKA2sv3, PHKA2sv4, PHKA2sv6.1, PHKA2sv6.2 and PHKA2sv7 splice variant mRNAs.

Clones having the PHKA2sv3 structure are identified based upon amplification of an amplicon band of about 345 basepairs, whereas a normal reference PHKA2 clone will give rise to an amplicon band of about 521 basepairs. Clones having the PHKA2sv4 structure are identified based upon amplification of an amplicon band of about 300 basepairs, whereas a normal reference PHKA2 clone would give rise to an amplicon band of about 447 basepairs. Clones having the PHKA2sv6.1 structure are identified based upon amplification of an amplicon band of about 396 basepairs, whereas a normal reference PHKA2 clone would not give rise to an amplicon band at all, because the SEQ ID NO 31 primer is located in intron 16 which is missing from the reference PHKA2 mRNA. Clones having the PHKA2sv6.2 structure are identified based upon amplification of an amplicon band of about 202 basepairs, whereas a normal reference PHKA2 clone would not give rise to an amplicon band at all, because the SEQ ID NO 32 primer is located in intron 16 which is missing from the reference PHKA2 mRNA. Clones having the PHKA2sv7 structure are identified based upon amplification of an amplicon band of 805 basepairs, whereas a normal reference PHKA2 clone would give rise to an amplicon band of 904 basepairs. DNA sequence analysis of the PHKA2sv3, PHKA2sv4, PHKA2sv6.1, PHKA2sv6.2 or PHKA2sv7 cloned DNAs produce a polynucleotide sequence having a PHKA2sv3, PHKA2sv4, PHKA2sv6.1, PHKA2sv6.1 or PHKA2sv7 coding sequence of SEQ ID NO 1, SEQ ID NO 3, SEQ ID NO 5, SEQ ID NO 7 or SEQ ID NO 9 respectively.

SEQ ID NO 1 contains an open reading frame that encodes a PHKA2sv3 protein (SEQ ID NO 2) similar to the reference PHKA2 protein (NP_{—}000283), but lacking a 203 base pair region encoded by exons 27, 28 and 29 of the full length coding sequence of reference PHKA2 mRNA (NM_{—}000292). The alternative splicing of the coding sequence of exons 27, 28 and 29, not only drops a 203 base pair region but also results in the creation of a protein translation reading frame that is out of alignment in the exon 30 nucleotide sequence in comparison to the reference PHKA2 protein reading frame. This shift in reading frame results in the production of an altered and shorter PHKA2sv3 protein as compared to the reference PHKA2 protein (NP_{—}000283). In particular, the last 24 amino acids of the PHKA2sv3 polypeptide are not present in reference PHKA2 (NP_{—}000283).

SEQ ID NO 3 contains an open reading frame that encodes a PHKA2sv4 protein (SEQ ID NO 4) similar to the reference PHKA2 protein (NP_{—}000283), but lacking a 145 nucleic acid region encoded by exon 16 of the full length coding sequence of reference PHKA2 mRNA (NM_{—}000292). The alternative splicing of the coding sequence of exon 16 not only drops a 145 base pairs region but also results in the creation of a protein translation reading frame that is out of alignment in comparison to the reference PHKA2 protein reading frame. This shift in reading frame results in the production of an altered and shorter PHKA2sv3 protein as compared to the reference PHKA2 (NP_{—}000283). In particular, the last 17 amino acids of the PHKA2sv3 polypeptide are not present in reference PHKA2 (NP_{—}000283).

SEQ ID NO 5 contains an open reading frame that encodes PHKA2sv6.1 protein (SEQ ID NO 6). The PHKA2sv6.1 polypeptide (SEQ ID NO 6) is similar to the reference PHKA2 protein (NP_{—}000283), but contains an additional 246 nucleic acid region encoded by intron 16 of the full length PHKA2 gene. The 246 nucleotide insertion includes a novel in frame stop codon, which results in the results in the creation of a truncated protein in comparison to the reference PHKA2 protein (NP_{—}00283). In particular, the last 22 amino acids of the PHKA2sv6.1 polypeptide are not present in reference PHKA2 (NP_{—}000283).

SEQ ID NO 7 contains an open reading frame that encodes PHKA2sv6.2 protein (SEQ ID NO 8). The PHKA2sv6.2 polypeptide (SEQ ID NO 8) is similar to the carboxy-terminus of the reference PHKA2 protein (NP_{—}000283), but contains an additional 246 nucleic acid region encoded by intron 16 of the full length PHKA2 gene. The 246 nucleotide insertion includes a novel in frame start codon, which results in the creation of an amino-terminal truncated protein in comparison to the reference PHKA2 protein (NP_{—}00283). In particular, the first 28 amino acids of PHKA2sv6.2 polypeptide are not present in reference PHKA2 (NP_{—}000283).

SEQ ID NO 9 contains an open reading frame that encodes a PHKA2sv7 protein (SEQ ID NO 10) similar to the reference PHKA2 protein (NP_{—}000283), but lacking a 99 nucleic acid region encoded by exon 7 of the full length coding sequence of reference PHKA2 mRNA (NM_{—}000292). The alternative splicing of the coding sequence of exon 7 results in the creation of a protein translation reading frame that is in alignment in comparison to the reference PHKA2 protein reading frame. This results in the production of a PHKA2sv7 protein that lacks 33 internal amino acids as compared to the reference PHKA2 (NP_{—}000283).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified nucleic acid comprising SEQ ID NO 9, or the complement thereof.

2. The purified nucleic acid of claim 1, wherein said nucleic acid comprises a sequence encoding SEQ ID NO 9.

3. The purified nucleic acid of claim 1, wherein said nucleotide sequence encodes a polypeptide consisting of SEQ ID NO 10.

4. A purified polypeptide comprising SEQ ID NO 10.

5. The polypeptide of claim 4, wherein said polypeptide consists of SEQ ID NO 10.

6. An expression vector comprising a nucleotide sequence encoding SEQ ID NO 10, wherein said nucleotide sequence is transcriptionally coupled to an exogenous promoter.

7. The expression vector of claim 6, wherein said nucleotide sequence comprises SEQ ID NO 9.

8. The expression vector of claim 6, wherein said nucleotide sequence consists of SEQ ID NO 9.

9. A method of screening for compounds that bind selectively to PHKA2sv7 comprising the steps of:
(a) providing a PHKA2sv7 polypeptide comprising SEQ ID NO 10;
(b) providing one or more PHKA2 isoform polypeptides that are not PHKA2sv7;
(c) contacting said PHKA2sv7 polypeptide and said PHKA2 polypeptide that is not PHKA2sv7 with a test preparation comprising one or more compounds; and
(d) determining the binding of said test preparation to said PHKA2sv7 polypeptide and to said PHKA2 polypeptide that is not PHKA2sv7, wherein a preparation that binds to said PHKA2sv7 polypeptide but does not bind to said PHKA2 polypeptide that is not PHKA2sv7 contains a compound that selectively binds to said PHKA2sv7 polypeptide.

10. The method of claim 9, wherein said PHKA2sv7 polypeptide is obtained by expression of said polypeptide from an expression vector comprising a polynucleotide encoding SEQ ID NO 9.

11. A method of screening for a compound able to bind a PHKA2sv7 or a fragment thereof comprising the steps of:
(a) expressing a PHKA2sv7 polypeptide comprising SEQ ID NO 10 or fragment thereof from recombinant nucleic acid;
(b) providing to said polypeptide a labeled PHKA2 ligand that binds to said polypeptide and a test preparation comprising one or more test compounds; and
(c) measuring the effect of said test preparation on binding of said labeled PHKA2 ligand to said polypeptide, wherein a test preparation that alters the binding of said labeled PHKA2 ligand to said polypeptide contains a compound that binds to or interacts with said polypeptide.

12. The method of claim 11, wherein said steps (b) and (c) are performed in vitro.

13. The method of claim 11, wherein said steps (a), (b) and (c) are preformed using a whole cell.

14. The method of claim 11, wherein said polypeptide is expressed from an expression vector.

15. The method of claim 14, wherein said expression vector comprises SEQ ID NO 9 or a fragment of SEQ ID NO 9.

16. The method of claim 11, wherein said test preparation contains one compound.

17. The method of claim 11, wherein said PHKA2 ligand is a phosphorylase kinase inhibitor.
